(19) United States
(12) Patent Application Publication
(10) Pub. No.: US 2022/0168384 A1
(43) Pub. Date: Jun. 2, 2022

(54) Anti-Bacterial Combination Therapy

(71) Applicant: McMaster University, Hamilton (CA)

(72) Inventors: Lori L. Burrows, Oakville (CA); Michael M.R. Ranieri, Toronto (CA); Derek C.K. Chan, Markham (CA); Luke N. Yaeger, Hamilton (CA)

(21) Appl. No.: 17/434,156

(22) PCT Filed: Feb. 26, 2020
(86) PCT No.: PCT/CA2020/050247

§ 371 Date: Aug. 26, 2021

Related U.S. Application Data
(60) Provisional application No. 62/810,645, filed on Feb. 26, 2019.

(51) Int. Cl. A61K 38/12; A61P 31/04; A61K 45/06; A61K 38/42; A61K 38/40

(52) U.S. Cl. CPC A61K 38/12; A61P 31/04; A61K 38/40; A61K 38/42; A61K 45/06

(57) ABSTRACT

Provided herein in aspects is an antibacterial combination comprising a thiopeptide antibiotic and an iron inhibitor. Methods of treatment and/or prevention of bacterial infections using the combination are also provided as well as methods of sensitizing a gram-negative bacteria to a thiopeptide antibiotic and methods of screening a molecule for antimicrobial activity.

### FIELD

The present invention relates to bacterial infections. More specifically, the present invention relates to combination treatment of bacterial infections and related compositions and methods.

### BACKGROUND

Bacterial pathogens are rapidly evolving resistance to available antibiotics, creating an urgent need for new therapies. Gram-negative bacteria are particularly challenging to treat because their outer membranes limit the access of many drugs to intracellular targets (1). Resistance arises when bacteria accumulate target mutations, acquire specific resistance determinants, increase drug efflux, and/or enter antibiotic-tolerant dormant or biofilm modes of growth (2). Biofilms consist of surface-associated bacteria surrounded by self-produced extracellular polymeric substances (EPS). Biofilm architecture allows for development of phenotypic heterogeneity that leads to variations in susceptibility as well as the formation of drug-tolerant persister cells (3). Approaches with the potential to preserve current antibiotics include combining them with biofilm inhibitors, resistance blockers (e.g. ampicillin with clavulanic acid or piperacillin with tazobactam), efflux inhibitors (e.g. PAPN), outer membrane permeabilizers, or coupling them to molecules such as siderophores that are actively imported, so-called Trojan horses (4).

Among the bacterial pathogens deemed most problematic by the World Health Organization is the Gram-negative opportunist, Pseudomonas aeruginosa (5). It infects immunocompromised patients—particularly those with medical devices—and is a major problem for people with severe burns or the genetic disorder, cystic fibrosis (6). It is intrinsically resistant to many antibiotics and readily forms biofilms, further enhancing its ability to evade therapy (7). The low permeability of its outer membrane and multiple efflux pumps that extrude a wide variety of substrates, coupled with its propensity to form biofilms, limits the repertoire of effective anti-Pseudomonas antibiotics (8-10).

### SUMMARY

In an aspect, compositions are provided comprising a thiopeptide antibiotic and an iron chelator.

In another aspect, a method of treating a bacterial infection in a mammal is provided. The method comprises administering to the mammal an effective amount of a thiopeptide antibiotic and an iron chelator.

In a further aspect, a method of treating an antimicrobial resistant bacterial infection in a mammal is provided. The method comprises administering to the mammal an effective amount of a thiopeptide antibiotic and an iron chelator.

In an aspect, an article of manufacture is provided. The article of manufacture comprises packaging material containing a composition. The composition comprises a thiopeptide antibiotic and an iron chelator. The packaging material is labeled to indicate that the composition is useful to treat a bacterial infection in a mammal.

In another aspect, a composition comprised of a thiopeptide antibiotic and an iron chelator for use in the treatment of a bacterial infection in a mammal is provided.

In accordance with an aspect, there is provided a combination comprising a thiopeptide antibiotic and an iron inhibitor.

In an aspect, the thiopeptide antibiotic is thiostrepton, siomycin A, thiocillin I, micrococcin P1, nosiheptide, berninamycin A, geninthiocin A, a derivative thereof, a prodrug thereof, a salt thereof, or a combination thereof.

In an aspect, the thiopeptide antibiotic is thiostrepton.

In an aspect, the iron inhibitor is an iron chelator or an iron analogue.

In an aspect, the iron inhibitor comprises deferiprone, deferasirox, deferoxamine, transferrin, hemoglobin, lactoferrin, doxycycline, ciclopirox olamine, tropolone, clioquinol, gallium nitrate, or a combination thereof.

In an aspect, the iron inhibitor comprises deferiprone, deferasirox, deferoxamine, or a combination thereof.

In an aspect, the iron inhibitor comprises deferasirox and doxycycline.

In an aspect, the thiopeptide antibiotic and the iron inhibitor are in synergistic amounts for treating and/or preventing a bacterial infection in a subject.

In an aspect, the combination comprises the thiopeptide antibiotic in an amount of from about 0.01 μM to about 1000 mM.

In an aspect, the thiopeptide antibiotic is for use in an amount of from about 1 to about 1000 mg/kg/day.

In an aspect, the combination comprises the iron inhibitor in an amount of from about 0.01 μM to about 1000 mM.

In an aspect, the iron inhibitor is for use in an amount of from about 1 to about 1000 mg/kg/day.

In an aspect, the bacterial infection is a gram-negative bacterial infection.

In an aspect, the bacterial infection is a Pseudomonas aeruginosa infection, an Acinetobacter baumannii infection, or a combination thereof.

In an aspect, the bacterial infection is a multi-drug resistant bacterial infection.

In an aspect, the bacterial infection is caused by a bacteria that expresses a siderophore receptor.

In an aspect, the bacterial infection is caused by a bacteria that expresses a type I pyoverdine receptor.

In an aspect, the type I pyoverdine receptor is FpvA, FpvB, a homolog thereof, or a combination thereof.

In accordance with an aspect, there is provided a composition comprising the combination described herein.

In an aspect, the composition is for oral, injectable, or topical use.

In an aspect, the composition is a topical composition formulated for example as a lotion, gel, spray, or ointment.

In accordance with an aspect, there is provided a kit comprising the combination described herein.

In accordance with an aspect, there is provided a method of treating and/or preventing a bacterial infection in a subject, the method comprising administering an effective amount of the combination, the composition, or the kit described herein to the subject.

In accordance with an aspect, there is provided a method of treating and/or preventing a gram-negative bacterial infection in a subject, the method comprising administering a thiopeptide antibiotic to the subject.

In an aspect, the thiopeptide antibiotic is thiostrepton, siomycin A, thiocillin I, micrococcin P1, nosiheptide, berninamycin A, geninthiocin A, a derivative thereof, a prodrug thereof, a salt thereof, or a combination thereof.

In an aspect, the thiopeptide antibiotic is thiostrepton.

In an aspect, the method further comprises administering an iron inhibitor to the subject.

In an aspect, the thiopeptide antibiotic and the iron inhibitor are administered simultaneously or sequentially.

In an aspect, the iron inhibitor is an iron chelator or an iron analogue.

In an aspect, the iron inhibitor comprises deferiprone, deferasirox, deferoxamine, transferrin, hemoglobin, lactoferrin, doxycycline, ciclopirox olamine, tropolone, clioquinol, gallium nitrate, or a combination thereof.

In an aspect, the iron inhibitor comprises deferiprone, deferasirox, deferoxamine, or a combination thereof.

In an aspect, the iron inhibitor comprises deferasirox and doxycycline.

In an aspect, the thiopeptide antibiotic and the iron inhibitor synergistically treat and/or prevent the bacterial infection.

In an aspect, the thiopeptide antibiotic is in an amount of from about 0.01 μM to about 1000 mM.

In an aspect, the thiopeptide antibiotic is administered in an amount of from about 1 to about 1000 mg/kg/day.

In an aspect, the iron inhibitor is in an amount of from about 0.01 μM to about 1000 mM.

In an aspect, the iron inhibitor is administered in an amount of from about 1 to about 1000 mg/kg/day.

In an aspect, the bacterial infection is a Pseudomonas aeruginosa infection, an Acinetobacter baumannii infection, or a combination thereof.

In an aspect, the bacterial infection is a multi-drug resistant bacterial infection.

In an aspect, the bacterial infection is caused by a bacteria that expresses a siderophore receptor.

In an aspect, the bacterial infection is caused by a bacteria that expresses a type I pyoverdine receptor.

In an aspect, the type I pyoverdine receptor is FpvA, FpvB, a homolog thereof, or a combination thereof.

In an aspect, the method is for oral, injectable, or topical administration.

In an aspect, the method is for topical administration of for example a lotion, gel, spray, or ointment.

In accordance with an aspect, there is provided a method of sensitizing a gram-negative bacteria to a thiopeptide antibiotic, the method comprising administering an iron inhibitor to the gram-negative bacteria.

In accordance with an aspect, there is provided a method of screening a molecule for antimicrobial activity, the method comprising measuring biofilm stimulation or a proxy thereof by the molecule, wherein stimulation of biofilm formation is suggestive that the molecule has antimicrobial activity.

In accordance with an aspect, there is provided a use of the combination, the composition, or the kit described herein for treating and/or preventing a bacterial infection in a subject.

In accordance with an aspect, there is provided the combination, the composition, or the kit described herein for use in treating and/or preventing a bacterial infection in a subject.

In accordance with an aspect, there is provided a use of a thiopeptide antibiotic for treating and/or preventing a gram-negative bacterial infection in a subject.

In accordance with an aspect, there is provided a thiopeptide antibiotic for use in treating and/or preventing a gram-negative bacterial infection in a subject.

In accordance with an aspect, there is provided a use of an iron inhibitor for sensitizing a gram-negative bacteria to a thiopeptide antibiotic.

In accordance with an aspect, there is provided an iron inhibitor for use in sensitizing a gram-negative bacteria to a thiopeptide antibiotic.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Certain embodiments will now be described in greater detail with reference to the drawings in which:

FIG. 1 shows thiostrepton stimulates P. aeruginosa biofilm formation. A) Structure of thiostrepton (TS). B) TS stimulated biofilm formation of P. aeruginosa PAO1 and decreased planktonic cell density in 10:90 medium in a dose-dependent manner, up to its maximum soluble concentration of 10 μM (17 μg/ml). C) In VBMM, PAO1 biofilm formation was stimulated by TS, while planktonic cell density dropped below the level of detection at concentrations above 0.63 μM. Assays were performed at least 3 times in triplicate; **** p<0.0001.

FIG. 2 shows growth of P. aeruginosa PAO1 in various media. The growth (OD₆₁₂) of strain PAO1 in various media was monitored for 24 h. Growth in 10:90 (10% LB, 90% PBS) was similar to that in VBMM (Vogel Bronner Minimal Medium). The experiment was performed 3 times in triplicate.

FIG. 3 shows thiostrepton susceptibility in Mueller-Hinton Broth versus 10:90. The susceptibility of P. aeruginosa PA14, E. coli BW25113, A. baumannii C0286, and S. aureus USA300 was compared in MHB versus 10:90 medium. E. coli lacks FpvAB and is resistant to TS in both media. S. aureus is highly susceptible to TS in both media, suggesting that TS uptake by Gram positive bacteria is independent of iron levels.

FIG. 4 shows expression of Tsr in trans reduces susceptibility of P. aeruginosa to thiostrepton. Expression of the tsr gene from Streptomyces azureus in trans from pUCP20 in two strains of P. aeruginosa reduced their susceptibility to TS in VBMM, suggesting it inhibits growth via its canonical mode of action, disrupting translation. A) Growth of PAO1; B) Growth of PA14. Each assay was performed at least 3 times in triplicate; **** p<0.0001.

FIG. 5 shows thiostrepton susceptibility is unaffected by addition of Mg⁺⁺ or casamino acids, or deletion of peptide transporters. A) To rule out increased susceptibility to TS due to enhanced outer membrane permeability from chelation of divalent cations by citrate—the carbon source in VBMM—the medium was supplemented with 100 mM MgCl₂. Growth and biofilm formation are plotted as percent of the DMSO control on a log₁₀ scale. B) Supplementation of VBMM with 0.1% (w/v) casamino acids did not affect susceptibility of PAO1 to TS. C) Deletion of the C and D components of peptide transport systems Opp (also called Npp) used by other peptide antibiotics including pacidamycin, blasticidin S, microcin C, and albomycin for uptake and a homologous system Spp, had no effect on TS susceptibility.

FIG. 6 shows thiostrepton activity is potentiated by iron chelators. Biofilm stimulation by TS in 10:90 medium increased in the presence of 0.1 μM EDDHA, a cell-impermeant iron chelator, while further addition of 100 μM FeCl₃ increased the concentration of TS required for biofilm stimulation and growth inhibition. PAO1 growth (OD₆₀₀) and biofilm (absorbance of CV at 600 nm) in A) 10:90 medium alone; B) 10:90 plus 0.1 μM EDDHA; C) 10:90 plus 0.1 μM EDDHA and 100 μM FeCl₃. Checkerboard assays showed that TS activity against PAO1 was potentiated by FDA approved iron chelators, D) deferiprone and E) deferasirox or by 10% heat-inactivated F) mouse serum or G) human serum. The highest concentration of DFP (3680 μM) and DSX (1370 μM) is each equal to 512 μg/ml. Each assay was performed at least 3 times; *** p<0.001; **** p<0.0001.

FIG. 7 shows susceptibility of P. aeruginosa efflux mutants to thiostrepton. Mutants of strain PAO1 in which the gene(s) encoding the outer membrane component of the major efflux systems MexAB-OprM (shared with MexXY), MexCD-OprJ, and MexGH-OpmD were deleted singly or in combination and tested for susceptibility to thiostrepton. There were no significant differences in susceptibility compared to the wild-type parent.

FIG. 8 shows checkerboard assays of various antibiotics with deferasirox (DSX). To test whether DSX could potentiate the activity of antibiotics besides thiostrepton against P. aeruginosa PA14, checkboard assays of DSX with A) doxycycline, B) ciprofloxacin, C) tobramycin, or D) chloramphenicol were performed. No synergy with these antibiotics was observed, suggesting that their activity is not potentiated by DSX.

FIG. 9 shows thiostrepton inhibits growth of multidrug resistant clinical isolates. The growth of most clinical isolates of A) P. aeruginosa and B) Acinetobacter baumannii resistant to multiple antibiotics was inhibited by 5 μM (8.3 μg/ml) TS in 10:90 medium (grey bars). TS activity was potentiated by the addition of 86 μM deferasirox (DSX; 32 μg/ml; black bars). Each assay was performed at least 3 times and the results plotted as percent of the DMSO-only growth control (OD₆₀₀) on a log₁₀ scale. Error bars equal standard deviation.

FIG. 10 shows Acinetobacter baumannii encodes homologs of P. aeruginosa FpvA and FpvB. The amino acid sequences of P. aeruginosa FpvA and FpvB were aligned with their closest homologs in A. baumannii using MUSCLE (Genious). The FpvA-Pa and FpvA-Ab homologs share ˜44% identity, while the FpvB homologs from the two species are nearly identical (99%). FpvA-Pa, PA2398; FpvB-Pa, PA4168; FpvA-Ab, GenBank: SSM88576.1; FpvB-Ab, GenBank: SCZ16661.1.

FIG. 11 shows thiostrepton does not bind iron. To determine whether the uptake of TS by pyoverdine receptors depends on formation of a ferric complex, its ability to decolorize chrome azurol S (CAS) agar was tested. A lack of a color change for TS indicates that in contrast to chelators deferiprone and deferasirox, it is unlikely to chelate iron. The glycopeptide antibiotic vancomycin was used as a negative control. Five microliters of each compound at 2 mg/ml was spotted onto CAS agar, and the plate was incubated at room temperature for 1 h.

FIG. 12 shows qualitative assay to identify potential antibiotic-Fe³⁺497 complexes. Binding of iron by a compound causes spectral shifts that can detected visually. Five μL of stock concentration antibiotic was added to 5 μL of FeCl₃ to a final FeCl₃ concentration of 10 μM and incubated at room temperature for one hour. Negative controls without iron are indicated by a negative sign and droplets with FeCl₃ are indicated with a positive sign; vehicle controls with Milli-Q H₂O and DMSO were included.

FIG. 13 shows UV-Vis absorption spectrum of compounds with and without Fe (III). Equimolar concentrations of compound and FeCl₃ were added in deionized H₂O to a final concentration of 300 μM and a spectrum of wavelengths from 300 nm to 700 nm read after 1 h incubation at room temperature. The black dashed line is the spectrum of the compound in the absence of iron. The black solid line is the spectrum after the addition of iron. New peaks appearing after the addition of Fe³⁺ are indicated with arrows. Chloramphenicol and ampicillin were used as negative controls. Each assay was performed at least 3 times and averaged values are shown.

FIG. 14 shows a chrome azurol S (CAS) assay of potential iron chelators. A representative plate is shown as an exemplary embodiment of the application. Doxycycline (DOXY), ciprofloxacin (CIP), deferasirox (DSX), clioquinol (CLI), tropolone (TRO), ciclopiroxolamine (CO), chloramphenicol (CHLOR), and ampicillin (AMP) were standardized to 2 mg/mL. Ten μL was spotted in each sector. The plate was incubated at room temperature for 1 h. CLI precipitated on the surface of the agar. DSX served as a positive control. CHLOR and AMP were negative controls. Decolourization was not observed for CLI due to precipitation.

FIG. 15 shows iron inhibitors are synergistic with TS against P. aeruginosa PA14. A) Checkerboards and B) IC₅₀ isobolograms are shown for each compound that synergize with TS. Dashed lines indicate the line of additivity and solid lines indicate the IC₅₀ of TS at each compound concentration. Checkerboards and IC₅₀ isobolograms are arranged in the following order: CO, CLI, TRO, DOXY, and GN from left to right, top to bottom. C) Combination indices (CI) of each TS combination. CI values are indicated at the top of the bars. All experiments were conducted 3 times. Average values are reported; **** p<0.0001, *** p<0.001, ** p<0.01.

FIG. 16 shows iron chelators antagonize gallium nitrate (GN). A) GN+DSX and B) GN+CO. DSX with GN has an additive effect at low concentrations but antagonizes at higher concentrations. The average of three independent experiments are shown for each checkerboard.

FIG. 17 shows addition of 100 μM FeCl₃ reduces the inhibitory activity of potential chelators. Vancomycin (VAN)+compound checkerboards are shown. None of the compounds synergized with VAN although an additive effect was seen with some compounds. The additive effect was abrogated by the addition of FeCl₃ but not MgCl₂ or CaCl₂, indicating that the effects of calcium and magnesium ion chelation from the outer membrane is minimal. The average of three independent repeats are shown for each checkerboard.

FIG. 18 shows the configuration and interpretation of 3D checkerboards. Three-dimensional checkerboards were arrayed in an 8×8×8 format (plate diagram created with BioRender). Each plate had identical serial dilutions of TS and DSX and a single concentration of the third compound. The direction of the arrows indicates increasing TS, DSX, or chelator concentration. Using MATLAB, each checkerboard was plotted in 3D with the % of control growth in the z-axis. The surface area of the checkerboard was calculated and expressed in terms of % of control, which was plotted against the third drug concentration. These data were compared to the dose-response curve of the third compound alone and the datasets compared using Graphpad Prism to identify statistically significant differences using 2-way ANOVA.

FIG. 19 shows DSX and putative iron chelators show no synergy against P. aeruginosa PA14. A) DSX+Clioquinol; B. DSX+CO); C) DSX+TRO and D) DSX+DOXY. DSX was additive to DOXY and indifferent with CO and TRO. DSX showed antagonism with CLI at the highest concentration tested. Higher concentrations were not tested because CLI precipitates above 8 μg/mL in aqueous media. The average of three independent experiments are shown for each checkerboard.

FIG. 20 shows unidirectional synergy between chelators and TS. Surface areas of 3D checkerboards were plotted against chelator concentration in term of % of control on a log 10 scale and compared to the activity of each chelator alone. A) DOXY; B) CO; C) CLI and D) TRO. E) Surface areas were graphed with respect to increasing TS concentrations and compared to the activity of TS alone. n.s., not significantly different; * p<0.05, ** p<0.005, *** p<0.0005. The average of at least three biological replicates are shown.

FIG. 21 shows TS combinations are bactericidal. Checkerboards were pinned to LB agar plates using a sterile 96 pin tool. Plates were incubated overnight at 37° C. Independent experiments were conducted 3 times and a representative plate is shown.

FIG. 22 shows TS combinations inhibit the growth of clinical isolates. Single, double, and triple TS combinations were used to inhibit the growth of A) P. aeruginosa and B) A. baumannii clinical isolates. Highly-resistant strain C0379 is plotted in grey. TS and DSX were used at 8.3 μg/mL and 32 μg/mL respectively. The third compound was used at ¼ MIC against PA14 (1 μg/mL DOXY, 2 μg/mL CO, 4 μg/mL TRO, and 1 μg/mL CLI). Horizontal bars show depict the mean % of control growth. Assays were performed at least 3 times. Averaged values are shown. Statistics for TS+DSX versus TS alone or versus other combinations are shown. n.s., not significantly different; * p<0.05; *** p<0.0005; **** p<0.0001.

FIG. 23 shows growth of highly-resistant strain C0379 is inhibited with increased concentrations of CO, CLI, and DOXY. The resistant clinical isolate was challenged with single, double, and triple combinations of A) CO; B) CLI; C) DOXY and D) TRO. Increasing doses of each compound were combined with TS and DSX at 8.3 μg/mL and 32 μg/mL, respectively. MIC assays were conducted at least 3 times and averaged results are shown; ** p<0.001.

FIG. 24 shows A. baumannii C0286 is susceptible to CLI at low concentrations. The average of 3 independent experiments is shown.

FIG. 25 shows structures of thiopeptides tested for synergy with DSX.

FIG. 26 shows checkerboard assays of thiopeptide+DSX against PA14. Assays were conducted in 10:90; results are averaged from three independent replicates.

FIG. 27 shows TC and TC+DSX dose-response assays against PA14 conducted in 10:90. Results were averaged from three independent replicates. not significant; ** p<0.005; *** p<0.0005; **** p<0.00005.

FIG. 28 shows TC and TC+DSX dose-response assays against various bacteria conducted in 10:90. A) WCC C0379; B) WCC C0286, C) S. typhimurium SL1344 and D) K. pneumoniae. Results are averaged from three independent replicates. n.s., not significant; * p<0.05; ** p<0.005; *** p<0.0005; **** p<0.00005.

FIG. 29 shows triple combination of TS+TC+DSX at sub-MIC reduces PA14 growth below the MIC. % of control growth less than 20% was considered as the MIC cut-off. Assays were conducted in VBMM. Results are averaged from three independent experiments; * p<0.05; ** p<0.005; *** p<0.0005.

### DETAILED DESCRIPTION

With the initial aim of identifying potential modulators of P. aeruginosa biofilm formation, a collection of bioactive molecules were screened, including previously FDA-approved off-patent drugs. During this work, several molecules were identified that stimulated biofilm formation beyond the arbitrary cutoff of 200% of the vehicle control. Investigation of one such stimulatory compound, thiostrepton (TS), revealed that it had low micromolar activity against P. aeruginosa in minimal medium. Through a series of investigations, they showed that TS gains access to its ribosomal targets by exploiting iron-dependent uptake pathways. These data show that the biofilm stimulation phenotype can reveal cryptic antibiotic activity when concentrations are too low (or growth conditions not conducive) to inhibit growth.

As described herein, TS susceptibility was inversely proportional to iron availability, suggesting that TS exploits uptake pathways whose expression is increased under iron starvation. Consistent with this finding, TS activity against P. aeruginosa and A. baumannii was potentiated by FDA-approved iron chelators, deferiprone and deferasirox. Screening of P. aeruginosa mutants for TS resistance revealed that it exploits pyoverdine receptors FpvA and FpvB to cross the outer membrane. The data show that the biofilm stimulation phenotype can reveal cryptic sub-inhibitory antibiotic activity, and that TS may be useful against select multidrug resistant Gram-negative pathogens under iron-limited growth conditions, similar to those encountered at sites of infection.

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

A “thiopeptide antibiotic” (also known as thiazolyl peptides) is a class of peptide antibiotics produced by bacteria that are sulfur-rich macrocyclic peptides containing highly-modified amino acids and characterized by a nitrogen-containing six-membered ring (such as piperidine, dehydropiperidine, or pyridine) substituted with multiple thiazole rings and dehydroamino acids. The macrocylic ring serves as a scaffold that also incorporates modified amino acids often with azole rings, such as thiazoles, oxazoles, and thiazolines. Some examples of thiopeptides include thiostrepton, cyclothiazomycin, nosiheptide, lactocillin, 554832A-I, MJ347-81F4A and B, and nocathiacins (Singh S B et al, 2013 J of Antibiotics 66:599-607), micrococcin P, nosiheptide (also known as multhiomycin), siomycin, sporangiomycin, althiomycin, the thiocillins and/or thiopeptin, as well as any other sulfur-rich peptide antibiotic containing multiple thiazole rings, produced by streptomycetes or other peptide antibiotic-producing organisms. Synthetic thiopeptide antibiotics are also contemplated.

An “iron inhibitor” is a compound that inhibits the action of iron. Examples of iron inhibitor include iron chelators and iron analogues. An “iron chelator” is a compound that acts as a chelating or binding agent for iron. Iron chelators can include any compound that has the appropriate molecular configuration to coordinate the binding of iron and/or other metals. Some iron chelators have been approved as drugs for the treatment of iron poisoning or chronic iron overload. These include FDA-approved compounds, deferoxamine, deferasirox and deferiprone. An “iron analogue” is a compound that mimics iron, such as gallium nitrate, and prevents or reduces the action of iron on a receptor.

The terms “treat,” “treating,” and “treatment” are used broadly herein to denote methods that at least reduce one or more adverse effects of a bacterial infection, including those that moderate or reverse the progression of, reduce the severity of, prevent, or cure the infection. The term “mammal” as it is used herein is meant to encompass humans as well as non-human mammals such as domestic animals (e.g. dogs, cats and horses), livestock (e.g. cattle, pigs, goats, sheep) and wild animals.

Effective dosage levels of the thiopeptide antibiotic and iron inhibitor compounds will vary with factors such as the mammal being treated, the compounds selected for use, and mode of administration. A “therapeutically effective dosage” of each of the thiopeptide antibiotic and iron inhibitor is a dosage that is effective to treat gram negative infections, for example, those caused by P. aeruginosa and/or A. baumannii.

The thiopeptide antibiotic and iron inhibitor may be administered via any suitable route. The antibiotic and iron inhibitor compounds may be administered together by the same or different route, or concurrently via the same or different routes. As will be appreciated by the skilled artisan, the route and/or mode of administration may vary on a number of factors, including for example, the compounds to be administered and the infection to be treated. Routes of administration include parental, such as intravenous, intramuscular, intradermal, intraperitoneal, subcutaneous, spinal, intraarterial, intrathecal, intracapsular, intraorbital, intracardiac, transtracheal, subcuticular, intraarticular, subcapsular, subarachnoid, intraspinal, epidural and intrasternal injection and infusion. Alternatively, non-parenteral routes may be used, including topical, epidermal or mucosal routes of administration, for example, intranasally, orally, vaginally, rectally, sublingually or topically.

Effective dosage levels of the thiopeptide antibiotic and iron inhibitor will vary with factors such as the mammal being treated, the compounds selected for use, and mode of administration. A “therapeutically effective dosage” of each of the thiopeptide antibiotic and iron inhibitor is a dosage that is effective to treat an infection caused by gram negative bacteria, including those caused by P. aeruginosa and A. baumannii.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

### Combinations

Described herein are various combinations comprising a thiopeptide antibiotic and an iron inhibitor. Any thiopeptide antibiotic and any iron inhibitor may be used in combination. Exemplary thiopeptide antibiotics include thiostrepton, siomycin A, thiocillin I, micrococcin P1, nosiheptide, berninamycin A, geninthiocin A, cyclothiazomycin, lactocillin, S54832A-J, MJ347-81F4A and B, nocathiacins, micrococcin P, sporangiomycin, althiomycin, the thiocillins and/or thiopeptin, as well as any other sulfur-rich peptide antibiotic containing multiple thiazole rings, produced by streptomycetes or other peptide antibiotic-producing organisms. Combinations of thiopeptide antibiotics are included herein. While a number of naturally occurring thiopeptide antibiotics have been listed, it will be understood that synthetic or partially synthetic thiopeptide antibiotics are also contemplated. Further, derivatives of thiopeptide antibiotics are included herein, including prodrugs and various salts or esters, for example. The thiopeptide antibiotics may be modified to improve their solubility, bioavailability, half-life, and so on, as will be understood by a skilled person. In typical aspects, the thiopeptide antibiotic is thiostrepton.

The iron inhibitor encompasses any molecule that inhibits the activity of iron at a receptor. For example, the iron inhibitor may be an iron chelator, which binds iron and prevents its activity directly, or an iron analogue, which in aspects competes with iron for receptor binding and prevents its activity indirectly. Exemplary iron inhibitors include deferiprone, deferasirox, deferoxamine, transferrin, hemoglobin, lactoferrin, doxycycline, ciclopirox olamine, tropolone, clioquinol, and gallium nitrate. As noted above with respect to thiopeptide antibiotics, derivatives of iron inhibitors are also contemplated as well as various combinations of different iron inhibitors. Typically, an iron chelator is not used in combination with an iron analogue, as the iron chelator will bind the analogue and reduce efficacy.

It will be understood that the thiopeptide antibiotic and the iron chelator are typically used in amounts that at least have an additive effect for treating and/or preventing a bacterial infection in a subject. Typically, however, they are used in synergistic amounts. For example, the thiopeptide antibiotic and/or the iron inhibitor may be present in the combination in an amount of from about 0.01 μM to about 100 mM, such as from about 0.01 μM, about 0.05 μM, about 0.1 μM, about 0.5 μM, about 1 μM, about 1.5 μM, about 2 μM, about 2.5 μM, about 3 μM, about 3.5 μM, about 4 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12.5 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 200 μM, about 300 μM, about 400 μM, about 500 μM, about 600 μM, about 700 μM, about 800 μM, about 900 μM, about 1 mM, about 10 mM, about 50 mM, about 100 mM, about 200 mM, about 300 mM, about 400 mM, about 500 mM, about 600 mM, about 700 mM, about 800 mM, or about 900 mM to about 0.05 μM, about 0.1 μM, about 0.5 μM, about 1 μM, about 1.5 μM, about 2 μM, about 2.5 μM, about 3 μM, about 3.5 μM, about 4 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12.5 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 200 μM, about 300 μM, about 400 μM, about 500 μM, about 600 μM, about 700 μM, about 800 μM, about 900 μM, about 1 mM, about 10 mM, about 50 mM, about 100 mM, about 200 mM, about 300 mM, about 400 mM, about 500 mM, about 600 mM, about 700 mM, about 800 mM, about 900 mM or about 1000 mM.

Similarly, the thiopeptide antibiotic and/or the iron inhibitor may be used in an amount of, for example, from about 1 mg/kg/day to about 1000 mg/kg/day, such as from about 1 mg/kg/day, about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 70 mg/kg/day, about 75 mg/kg/day, about 80 mg/kg/day, about 85 mg/kg/day, about 90 mg/kg/day, about 95 mg/kg/day, about 100 mg/kg/day, about 150 mg/kg/day, about 200 mg/kg/day, about 250 mg/kg/day, about 300 mg/kg/day, about 350 mg/kg/day, about 400 mg/kg/day, about 450 mg/kg/day, about 500 mg/kg/day, about 550 mg/kg/day, about 600 mg/kg/day, about 650 mg/kg/day, about 700 mg/kg/day, about 750 mg/kg/day, about 800 mg/kg/day, about 850 mg/kg/day, about 900 mg/kg/day, or about 950 mg/kg/day to about 5 mg/kg/day, about 10 mg/kg/day, about 15 mg/kg/day, about 20 mg/kg/day, about 25 mg/kg/day, about 30 mg/kg/day, about 35 mg/kg/day, about 40 mg/kg/day, about 45 mg/kg/day, about 50 mg/kg/day, about 55 mg/kg/day, about 60 mg/kg/day, about 70 mg/kg/day, about 75 mg/kg/day, about 80 mg/kg/day, about 85 mg/kg/day, about 90 mg/kg/day, about 95 mg/kg/day, about 100 mg/kg/day, about 150 mg/kg/day, about 200 mg/kg/day, about 250 mg/kg/day, about 300 mg/kg/day, about 350 mg/kg/day, about 400 mg/kg/day, about 450 mg/kg/day, about 500 mg/kg/day, about 550 mg/kg/day, about 600 mg/kg/day, about 650 mg/kg/day, about 700 mg/kg/day, about 750 mg/kg/day, about 800 mg/kg/day, about 850 mg/kg/day, about 900 mg/kg/day, about 950 mg/kg/day, or about 1000 mg/kg/day.

It will of course be understood that any given amounts may be varied depending on the potency of the particular thiopeptide antibiotic and iron inhibitor chosen for the combination in question. It is within the purview of the skilled person to test various combinations as described herein and modify the dosages as desired and test for synergy or additivity using the assays exemplified and described below.

The combinations described herein are typically used for treating a bacterial infection, such as a gram-negative bacterial infection. As it has been found that thiostrepton interacts with siderophore receptors, such as pyoverdine receptors, it will be understood that in typical aspects, the bacterial infection is caused by a bacteria that expresses siderophore receptors, such as pyoverdine receptors, such as type I pyoverdine receptors. Typically the type I pyoverdine receptor is FpvA or FpvB, as expressed in Pseudomonas aeruginosa or various homolog thereof that are expressed in other species. Combinations of these siderophore or pyoverdine receptors or homologs thereof may also be expressed by the bacteria.

Typically, the bacterial infection is caused by Pseudomonas aeruginosa and/or Acinetobacter baumannii. In certain aspects, the bacteria may be multi-drug resistant.

The combinations described herein may be provided in a single composition or the components may be provided separately in a kit, for example, or simply as two (or more) separate components. When used separately, they can be administered substantially simultaneously or sequentially in any order. The singular composition or separate components may be provided in any known form, such as a tablet, capsule, injection, inhalant, or topical, for example, as described in more detail below. Typically, the combination is for topical use and is formulated as one or more combined or separate creams, lotions, gels, sprays, or ointments.

The present combination may additionally include one or more pharmaceutically acceptable adjuvants or carriers. The expression “pharmaceutically acceptable” means acceptable for use in the pharmaceutical arts, i.e. not being unacceptably toxic, or otherwise unsuitable for administration to a mammal. Examples of pharmaceutically acceptable adjuvants include, but are not limited to, diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition.

In one embodiment, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution.

Compositions for oral administration via tablet, capsule, lozenge, solution or suspension in an aqueous or non-aqueous liquid, an oil-in-water or water-in-oil liquid emulsion, an elixir or syrup are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; tale; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, disintegrating agents, anti-oxidants, preservatives, colouring agents and favouring agents may also be present.

In another embodiment, the combination may be formulated for application topically as a cream, lotion, spray, gel, or ointment. For such topical application, the composition may include an appropriate base such as a triglyceride base and may also contain a surface-active agent and other cosmetic additives such as skin softeners and the like as well as fragrance. In some aspects, intended for topical application to an infected wound or burn, the combination may be infused in a wound dressing, for example.

Aerosol or other inhalable formulations, for example, for nasal delivery, may also be prepared in which suitable propellant adjuvants are used. The combinations described herein may also be administered as a bolus, electuary, or paste. Compositions for mucosal administration are also encompassed, including oral, nasal, rectal or vaginal administration for the treatment of infections, which affect these areas. Such compositions generally include one or more suitable non-irritating excipients or carriers comprising, for example, cocoa butter, polyethylene glycol, a suppository wax, a salicylate or other suitable carriers. Other adjuvants may also be added to the composition regardless of how it is to be administered, which, for example, may aid to extend the shelf-life thereof.

The combination of a thiopeptide antibiotic with an iron inhibitor as defined herein in aspects advantageously provides a synergistic anti-bacterial composition, i.e. a composition that exhibits activity that is greater than the expected additive activity of the thiopeptide antibiotic with the iron inhibitor. The thiopeptide antibiotic alone exhibits limited inhibitory activity against target bacteria, especially gram-negative bacteria while the iron inhibitor alone also exhibits little or no inhibitory activity against these bacteria. However, in combination, the thiopeptide antibiotic and iron inhibitor synergistically inhibit gram negative gram bacteria, including P. aeruginosa and A. baumannii, and associated antimicrobial-resistant strains.

### Methods of Use

Also described herein are various methods of use. For example, provided herein is a method of treating and/or preventing a bacterial infection in a subject. The method comprises administering an effective amount of a combination comprising a thiopeptide antibiotic and an iron inhibitor, as described above, to a subject in need thereof.

In further aspects, also described herein is a method of treating and/or preventing a gram-negative bacterial infection in a subject. In this aspect, the method comprises administering a thiopeptide antibiotic to the subject. Optionally, the method further comprises administering an iron inhibitor to the subject. As has been described above, the thiopeptide antibiotic and the iron inhibitor may be administered simultaneously or sequentially and can be any known thiopeptide antibiotic or iron inhibitor, examples of which are described above.

Typically, the thiopeptide antibiotic and the iron inhibitor synergistically treat and/or prevent the bacterial infection and these are used in amounts of from about 0.01 μM to about 100 μM, and/or from about 1 to about 1000 mg/kg/day. Typically, the bacterial infection is a Pseudomonas aeruginosa infection, an Acinetobacter baumannii infection, or a combination thereof. Treatment and/or prevention of a multi-drug resistant bacterial infection is contemplated herein. Typically, the bacteria that causes the bacterial infection expresses a type I pyoverdine receptor, such as FpvA, FpvB, a homolog thereof, or a combination thereof.

In further aspects, also described herein is a method of sensitizing a gram-negative bacteria to a thiopeptide antibiotic. In this aspect, the method comprises administering an iron inhibitor to the gram-negative bacteria. This will render the bacteria more sensitive to the thiopeptide antibiotic.

In further aspects, also described herein is a method of screening a molecule for antimicrobial activity. It has been described below that biofilm stimulation can be an indicator that a particular molecule may have antimicrobial activity and, thus, the method typically comprises measuring the ability of the molecule to stimulate biofilm production (or a proxy thereof). This can be much faster and/or easier than measuring MIC.

The above disclosure generally describes the present invention. A more complete understanding can be obtained by reference to the following specific examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

### EXAMPLES

### Example 1. A Combination of Thiostrepton and Known Iron Chelators are Effective in Inhibiting P. aeruginosa and Acinetobacter baumanii

In a high-throughput screen for molecules that modulate biofilm formation, it has been found that the thiopeptide antibiotic, thiostrepton (TS)—considered inactive against Gram-negative bacteria—stimulated P. aeruginosa biofilm formation in a dose-dependent manner. This phenotype is characteristic of exposure to antimicrobial compounds at sub-inhibitory concentrations, suggesting that TS was active against P. aeruginosa. Supporting this observation, TS inhibited growth of a panel of 96 multidrug-resistant (MDR) P. aeruginosa clinical isolates at low micromolar concentrations. TS also had activity against Acinetobacter baumannii clinical isolates and is expected to be generally effective against Gram-negative bacteria.

### Methods

### BACTERIAL Strains and Culture Conditions

The bacterial strains and plasmids used in this study are listed in Table 1 and Table 2. Bacterial cultures were grown in Lysogeny Broth (LB), 10:90 (10% LB and 90% phosphate buffered saline), M9 medium, Vogel Bonner minimal medium (VBMM), or cation-adjusted Mueller-Hinton broth (MBH) as indicated. Where solid media were used, plates were solidified with 1.5% agar. DFP (Sigma-Aldrich) and DSX (Cayman Chemicals) were stored at 4° C. until use. TS was stored at −20° C. A 60 mg/mL stock solution of DFP was made in 6M HCl and Milli-Q H₂O (DFP solvent) in a ratio of 3:50. A 20 mg/mL stock solution of DSX was made in DMSO. A 20 mM stock solution of TS was made in DMSO.

TABLE 1
Thiostrepton susceptibility of select P. aeruginosa mutants in VBMM.

| | | | IC_{CO} | MIC |
|---|---|---|---|---|
| Strain Name | PA Gene # | Protein(s) | (μg/mL) | (μM) |
| PA14 (wild type) | n/a | n/a | 0.26 | 0.60 |
| fpvA::Mar2xT7 | PA2398 | Type 1 ferripyoverdine receptor | 3.7 | 7.5 |
| fpvB::Mar2xT7 | PA4168 | Alternate Type 1 ferripyoverdine | 1.8 | 1.3 |
| | | receptor | | |
| pvdA::Mar2xT7 | PA2386 | L-ornithine N5-oxygenase | 0.26 | 0.21 |
| fpvA fpvB::Mar2xT7 | PA2398, | Type I ferripyoverdine receptor, | | |
| | PA4168 | Alternate type I ferripyoverdine | 6.9 | 7.5 |
| | | receptor | | |
| fpvA fpvB::Mar2xT7 + | PA2398, | Type I ferripyoverdine receptor, | 4.2 | 7.5 |
| pUCP20 | PA4168 | Alternate type I ferripyoverdine | | |
| | | receptor | | |
| fpvA fpvB::Mar2xT7 + | PA2398, | Type I ferripyoverdine receptor, | 2.8 | 2.5 |
| pUCP20-fpvB | PA4168 | Alternate type I ferripyoverdine | | |
| | | receptor | | |
| fptA::Mar2xT7 | PA4221 | Pyochelin receptor | 0.26 | 0.45 |
| PA1322::Mar2xT7 | PA1322 | Probable TonB-dependent receptor | 0.26 | 0.41 |
| PA4837::Mar2xT7 | PA4837 | Probable outer membrane protein | 0.26 | 0.41 |
| | | precursor | | |
| foxA::Mar2xT7 | PA2466 | Ferrioxamine receptor FoxA | 0.26 | 0.60 |
| fiuA::Mar2xT7 | PA0470 | Ferrichrome receptor FiuA | 0.26 | 0.36 |
| pirA::Mar2xT7 | PA0931 | Alternate enterobactin receptor | 0.35 | 0.60 |
| pfeA::Mar2xT7 | PA2688 | Enterobactin receptor | 0.26 | 0.82 |
| hasR::Mar2xT7 | PA3408 | Heme uptake outer membrane receptor | 0.35 | 0.60 |
| fithA::Mar2xT7 | PA4516 | Vibriobactin receptor | 0.26 | 0.50 |
| piuA::Mar2xT7 | PA4514 | Hydroxamate-type ferrisiderophore | 0.35 | 0.36 |
| | | receptor | | |
| PA0151::Mar2xT7 | PA0151 | Probable TonB-dependent receptor | 0.26 | 0.45 |
| chtA::Mar2xT7 | PA4675 | Aerobactin, Rhizobactin 1021, | 0.59 | 0.36 |
| | | Schizokinen receptor | | |
| phuR::Mar2xT7 | PA4710 | Heme/Hemoglobin uptake receptor | 0.26 | 0.21 |
| | | precursor | | |

TABLE 2
Antibiotic resistance of clinical Pseudomonas aeruginosa and Acinetobacter baumannii
strains.

| | |
|---|---|
| Strain | |
| number | Resistant to:^{a} |

P. aeruginosa clinical isolates (Wright Clinical Collection^{b})

| | |
|---|---|
| C0007 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0028 | ampicillin, amoxicillin clavulanic acid, cefazolin, cefalotin, ciprofloxacin, cefixime, |
| | metropenem, nitrofuratoin, tetracycline, trimethoprim sulfamethoxazole, cefoxitin, |
| | ceftriaxone |
| C0029 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0030 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0031 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0042 | ampicillin, amoxicillin clavulanic acid, cefazolin, cefalotin, nitrofuratoin, |
| | tetracycline, trimethoprim sulfamethoxazole, cefoxitin, ceftriaxone |
| C0043 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0053 | cefazolin, cefalotin, cefixime, nitrofuratoin, tetracycline |
| C0056 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0057 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0060 | ampicillin, amoxicillin clavulanic acid, cefazolin, cefalotin, ciprofloxacin, cefixime, |
| | gentamicin, nitrofuratoin, tetracycline, trimethoprim sulfamethoxazole, cefoxitin, |
| | ceftriaxone |
| C0062 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0063 | ampicillin, amoxicillin clavulanic acid, cefazolin, cefalotin, cefixime, ceftazidime, |
| | metropenem, nitrofuratoin, piperacillin tazobactam, tetracycline, trimethoprim |
| | sulfamethoxazole, cefoxitin, ceftriaxone |
| C0070 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0071 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0072 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0073 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0080 | nitrofuratoin, tetracycline |
| C0089 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0090 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0091 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0098 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0099 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0100 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0129 | ampicillin, amoxicillin clavulanic acid, cefazolin, cefalotin, cefixime, nitrofuratoin, |
| | tetracycline, cefoxitin, ceftriaxone |
| C0138 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0139 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0148 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0155 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0156 | ampicillin, amoxicillin clavulanic acid, cefazolin, cefalotin, cefixime, nitrofuratoin, |
| | piperacillin tazobactam, tetracycline, trimethoprim sulfamethoxazole, cefoxitin, |
| | ceftriaxone |
| C0157 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0166 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0167 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0168 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0176 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0177 | ampicillin, amoxicillin clavulanic acid, cefazolin, cefalotin, ciprofloxacin, cefixime, |
| | metropenem, nitrofuratoin, piperacillin tazobactam, tetracycline, trimethoprim |
| | sulfamethoxazole, cefoxitin, ceftriaxone |
| C0178 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0179 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0189 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0190 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0200 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0201 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0202 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0203 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0204 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0213 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0214 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0215 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0222 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0223 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0224 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0225 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0231 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0232 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0233 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0234 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0245 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0254 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0260 | ampicillin, amoxicillin clavulanic acid, cefazolin, cefalotin, cefixime, nitrofuratoin, |
| | piperacillin tazobactam, tetracycline, trimethoprim sulfamethoxazole, cefoxitin, |
| | ceftriaxone |
| C0261 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0262 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0263 | ampicillin, amoxicillin clavulanic acid, cefazolin, cefalotin, cefixime, metropenem, |
| | nitrofuratoin, tetracycline, trimethoprim sulfamethoxazole, cefoxitin, ceftriaxone |
| C0274 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0275 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0276 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0284 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0292 | ampicillin, amoxicillin clavulanic acid, cefazolin, cefalotin, ciprofloxacin, cefixime, |
| | ceftazidime, metropenem, nitrofuratoin, piperacillin tazobactam, tetracycline, |
| | trimethoprim sulfamethoxazole, cefoxitin, ceftriaxone |
| C0293 | ampicillin, amoxicillin clavulanic acid, cefazolin, cefalotin, cefixime, nitrofuratoin, |
| | piperacillin tazobactam, tetracycline, trimethoprim sulfamethoxazole, cefoxitin, |
| | ceftriaxone |
| C0294 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0295 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0296 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0305 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0306 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0307 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0315 | ampicillin, amoxicillin clavulanic acid, cefazolin, cefalotin, cefixime, nitrofuratoin, |
| | piperacillin tazobactam, tetracycline, trimethoprim sulfamethoxazole, cefoxitin, |
| | ceftriaxone |
| C0332 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0333 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0334 | ampicillin, amoxicillin clavulanic acid, cefazolin, cefalotin, cefixime, ceftazidime, |
| | metropenem, nitrofuratoin, piperacillin tazobactam, tetracycline, trimethoprim |
| | sulfamethoxazole, cefoxitin, ceftriaxone |
| C0335 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0344 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0345 | ampicillin, amoxicillin clavulanic acid, cefazolin, cefalotin, cefixime, nitrofuratoin, |
| | piperacillin tazobactam, tetracycline, trimethoprim sulfamethoxazole, cefoxitin, |
| | ceftriaxone |
| C0346 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0355 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0356 | ampicillin, amoxicillin clavulanic acid, cefazolin, cefalotin, cefixime, ceftazidime, |
| | nitrofuratoin, piperacillin tazobactam, tetracycline, trimethoprim sulfamethoxazole, |
| | cefoxitin, ceftriaxone |
| C0365 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0366 | ampicillin, amoxicillin clavulanic acid, cefazolin, cefalotin, cefixime, nitrofuratoin, |
| | piperacillin tazobactam, tetracycline, trimethoprim sulfamethoxazole, cefoxitin, |
| | ceftriaxone |
| C0376 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0377 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0378 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0379 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0398 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0399 | ampicillin, amoxicillin clavulanic acid, cefazolin, cefalotin, ciprofloxacin, cefixime, |
| | nitrofuratoin, piperacillin tazobactam, tetracycline, trimethoprim sulfamethoxazole, |
| | cefoxitin, ceftriaxone |
| C0400 | ampicillin, amoxicillin clavulanic acid, cefazolin, cefalotin, ciprofloxacin, cefixime, |
| | nitrofuratoin, tetracycline, trimethoprim sulfamethoxazole, cefoxitin, ceftriaxone |
| C0401 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0409 | ampicillin, amoxicillin clavulanic acid, cefazolin, cefalotin, cefixime, nitrofuratoin, |
| | tetracycline, trimethoprim sulfamethoxazole, cefoxitin, ceftriaxone |
| C0410 | ampicillin, amoxicillin clavulanic acid, cefazolin, cefalotin, ciprofloxacin, cefixime, |
| | metropenem, nitrofuratoin, tetracycline, trimethoprim sulfamethoxazole, cefoxitin, |
| | ceftriaxone |

Acinetobacter baumannii clinical isolates (Wright Clinical Collection)

| | |
|---|---|
| C0015 | ampicillin, amoxicillin clavulanic acid, cefazolin, cefalotin, cefixime, nitrofuratoin, |
| | cefoxitin |
| C0044 | ampicillin, amoxicillin clavulanic acid, cefazolin, cefalotin, cefixime, nitrofuratoin, |
| | cefoxitin, ceftriaxone |
| C0074 | ampicillin, amoxicillin clavulanic acid, amikacin, cefazolin, cefalotin, ciprofloxacin, |
| | cefixime, ceftazidime, gentamicin, metropenem, nitrofuratoin, tetracycline, tobramycin, |
| | trimethoprim sulfamethoxazole, cefoxitin, ceftriaxone |
| C0083 | ampicillin, amoxicillin clavulanic acid, cefazolin, cefalotin, cefixime, nitrofuratoin, |
| | cefoxitin |
| C0092 | ampicillin, amoxicillin clavulanic acid, amikacin, cefazolin, cefalotin, ciprofloxacin, |
| | cefixime, ceftazidime, gentamicin, metropenem, nitrofuratoin, tetracycline, tobramycin, |
| | trimethoprim sulfamethoxazole, cefoxitin, ceftriaxone |
| C0102 | ampicillin, amoxicillin clavulanic acid, cefazolin, cefalotin, cefixime, nitrofuratoin, |
| | tetracycline, cefoxitin |
| C0170 | ampicillin, amoxicillin clavulanic acid, cefazolin, cefalotin, cefixime, nitrofuratoin, |
| | trimethoprim sulfamethoxazole, cefoxitin |
| C0171 | ampicillin, amoxicillin clavulanic acid, cefazolin, cefalotin, cefixime, nitrofuratoin, |
| | cefoxitin |
| C0267 | ampicillin, amoxicillin clavulanic acid, cefazolin, cefalotin, cefixime, nitrofuratoin, |
| | cefoxitin, ceftriaxone |
| C0286 | ampicillin, amoxicillin clavulanic acid, amikacin, cefazolin, cefalotin, ciprofloxacin, |
| | cefixime, ceftazidime, gentamicin, metropenem, nitrofuratoin, tetracycline, tobramycin, |
| | trimethoprim sulfamethoxazole, cefoxitin, ceftriaxone |

^{a}based on CLSI breakpoints for P. aeruginosa or A. batunannii, respectively
^{b}the Wright Clinical Collection is an internal collection of clinical isolates sourced from Hamilton, ON hospitals in the last 2 years. Patient identifiers for these strains were removed to comply with privacy requirements and strains assigned local reference numbers.

### Growth Curves

PAO1-KP was inoculated from a −80° C. stock into 5 ml LB broth and grown with shaking at 200 rpm, 16 h, 37° C. The overnight culture was subcultured at 1:500 into 5 different media (LB, 10:90, M9, Mueller-Hinton (MH), and VBMM)—incubated at 37° C. for 6 h with shaking at 200 rpm. Each subculture was standardized to OD₆₀₀˜0.1 (Biomate 3 Spectrophotometer) then diluted 1:500 into the same medium. Six replicates of 200 μl of each sample were added to a 96 well plate, which was incubated at 37° C. for 24 h with shaking at 200 rpm (Tecan Ultra Evolution plate reader). The OD₆₁₂ was read every 15 min for 24 h. The data for the six replicates of each sample were averaged and the experiment was repeated 3 times. The final data with standard deviations were plotted using Prism (Graphpad).

### Biofilm Modulation Assay

Briefly, P. aeruginosa was inoculated in 5 mL of LB and grown at 37° C. overnight, shaking at 200 rpm, and subsequently standardized to an OD₆₀₀ of ˜ 0.1 in 10:90. For the initial screen, 1 mM compound stocks in DMSO were diluted 1:100 in standardized cell suspension (1.5 μL of compound stock in 148.5 μL of cell suspension) to a final concentration of 10 μM. Control wells contained 10:90 plus 1% DMSO (sterility control) or standardized cell suspension plus 1% DMSO (growth control). Biofilms were formed on polystyrene peg lids (Nunc). After placement of the peg lid, the plate was sealed with parafilm to prevent evaporation and incubated for 16 h at 37° C., 200 rpm. Following incubation, the 96-peg lid was removed and planktonic density in the 96 well plate measured at OD₆₀₀ to assess the effect of test compounds on bacterial growth. The lid was transferred to a new microtiter plate containing 200 μl of 1× phosphate-buffered saline (PBS) per well for 10 min to wash off any loosely adherent bacterial cells, then to a microtiter plate containing 200 μL of 0.1% (wt/vol) crystal violet (CV) per well for 15 min. Following staining, the lid was washed with 70 mL of dH₂O, in a single well tray, for 10 min. This step was repeated four times to ensure complete removal of excess CV. The lid was transferred to a 96-well plate containing 200 μL of 33% (vol/vol) acetic acid per well for 5 min to elute the bound CV. The absorbance of the eluted CV was measured at 600 nm (BioTek ELx800), and the results plotted as percent of the DMSO control using Prism (Graphpad). Screens were performed in duplicate. Compounds that resulted in <50% of control biofilm were defined as biofilm inhibitors, while compounds that resulted in >200% of control biofilm were defined as biofilm stimulators. Compounds of interest were further evaluated using the same assay but over a wider range of concentrations (dose-response assay). For all biofilm stimulation and growth inhibition assays, no turbidity was visible below 20% of the control.

For TS dose response assays, TS stock solutions were diluted in DMSO and 2 μL of the resulting solutions plus 148 μL of a bacterial suspension standardized to an OD₆₀₀ of ˜0.1 in 10:90 were added to a 96 well plate in triplicate, as described above. Control wells contained 148 μL of 10:90+1.3% DMSO (sterility control) or standardized bacterial suspension+1.3% DMSO (growth control). For ethylenediamine-N,N′-bis(2-hydroxyphenylacetic acid) (EDDHA) alone or with FeCl₃ experiments, 2 μL of each were added as aqueous solutions to reach final concentrations of 0.1 μM EDDHA and 100 μM FeCl₃, and the amount of bacterial suspension adjusted to keep the total well volume at 150 μL. Controls for EDDHA and FeCl₃ were 2 μL of sterile dH₂O. Biofilms were grown for 16 h at 37° C., 200 rpm, then stained and quantified as described above. Assays were performed in triplicate and results were graphed using Prism (Graphpad) as a percentage of the DMSO control.

### Compounds Screened

The biofilm modulation assay was used to screen the McMaster Bioactives compound collection. This curated collection includes off-patent, FDA-approved drugs from the Prestwick Chemical Library (Prestwick Chemical, Illkirch, France), purified natural products from the Screen-Well Natural Products Library (Enzo Life Sciences, Inc., Farmingdale, N.Y., USA), drug-like molecules from the Lopac¹²⁸⁰ (International Version) collection (Sigma-Aldrich Canada Ltd., Oakville, ON, Canada) and the Spectrum Collection (MicroSource Discovery Systems, Inc., Gaylordsville, Conn., USA) which includes off patent drugs, natural products, and other biologically active compounds. In total, the collection is 3921 unique compounds.

Construction of a Tsr Plasmid for Expression in P. aeruginosa

The tsr gene from pIJ6902 was PCR-amplified using primers 5′GAATCCCGGGCGGTAGGACGACCATGAC-3′ and 5′CTTCAAGCTTTTATCGGTTGGCCGCGAG-3′. Both the PCR product and pUCP20 vector were digested with SmaI and HindIII, gel-purified, and ligated at a 1:3 molar ratio using T4 DNA ligase. The ligated DNA was transformed into E. coli DH5a and transformants selected on LB agar containing 100 μg/mL ampicillin and 5-bromo-4-chloro-3-indolyl-β-D-galactopyranoside for blue-white selection. Plasmids from white colonies were purified using a GeneJet Plasmid Miniprep kit (Thermo Scientific) following the manufacturer's protocols. After verification by restriction digest and DNA sequencing, pUCP20 and pUCP20-tsr were each introduced into P. aeruginosa PAO1 and PA14 by electroporation. Transformants were selected on LB agar containing 200 μg/mL carbenicillin.

### Serum Preparation

Human serum (Corning) and mouse serum (Equitech-Bio) were stored at −20° C. Sera were aliquoted into 5-ml culture tubes by thawing once at 37° C. for 30 min with occasional gentle mixing. Culture tubes were frozen at −20° C. until use. To make 10% serum solutions, serum was thawed for 10 min at 37° C. and then heat inactivated at 57° C. for 30 min. Two milliliters of heat-inactivated serum were added to 18 ml of 10:90 medium and gently mixed. This 10% serum solution was used for checkerboard assays.

### MIC and Checkboard Assays

MICs were determined with microbroth dilution assays in Nunc 96-well plates. Vehicle controls consisted of 1:75 dilutions of DMSO in 10:90 inoculated with PA14 or its mutants as described in “Growth Curves” above. Sterile controls consisted of 1:75 dilutions of DMSO in 10:90. Seven serially diluted concentrations of TS—with 17 μg/mL being the highest final concentration—was set up in triplicate. Tests were done with 1:75 dilutions of each TS concentration in 10:90 inoculated with PA14 or its mutants as described in Growth Curves. Plates were sealed to prevent evaporation and incubated with shaking at 200 rpm, 16 h, 37° C. The OD₆₀₀ of the plates was read (Multiskan Go—Thermo Fisher Scientific) and used to calculate MIC. Growth is proportional to the darkness intensity whereas white indicates no observable growth. The final volume of each well was 150 μL and each experiment was repeated at least three times.

Checkerboard assays were set-up using Nunc 96-well plates in an 8-well by 8-well format. Two columns were allocated for vehicle controls and two columns for sterility controls. Vehicle controls contained 2 μL DMSO plus 2 μL DFP solvent for checkerboards with TS and DFP or 4 μL DMSO for TS and DSX, in 146 μL of 10:90 medium or 10% serum inoculated with PA14 or PAO1-KP as described in Growth Curves. Sterile controls contained the same components in 10:90 medium or 10% serum, without cells. Serial dilutions of TS—with 17 μg/mL being the highest final concentration—were added along the y axis of the checkerboard (increasing concentration from bottom to top) whereas serial dilutions of DFP or DSX—with 512 μg/mL being the highest final concentration—were added along the x axis (increasing concentration from left to right). The final volume of each well was 150 μL and each checkerboard was repeated at least three times. Plates were incubated and the final OD₆₀₀ determined as detailed above.

### Clinical Isolates Testing

Clinical isolates of P. aeruginosa and A. baumannii were inoculated from −80° C. stocks into 200 μL LB broth and grown with shaking at 200 rpm, 16 h, 37° C. in Nunc 96-well plates. The overnight cultures were subcultured (1:25 dilution) into 10:90 and grown with shaking at 200 rpm, 2 h, 37° C. Vehicle controls consisted of 4 μL of DMSO, 144 μL 10:90 and 2 μL of subculture. Sterile controls consisted of 4 μL of DMSO and 146 μL 10:90. Test samples consisted of 2 μL of TS (final concentration of 5 μM, 8.3 μg/ml), 2 μL of DMSO (or DSX, final concentration of 86 μM, 32 μg/mL), 144 μL 10:90 and 2 μL of subculture. The final volume of each well was 150 μL and each checkerboard was repeated at least three times. Plates were incubated with shaking at 200 rpm, 16 h, 37° C. and OD₆₀₀ was measured (Multiskan Go—Thermo Fisher Scientific). The results were plotted as percent of control (wells containing only DMSO) using Prism (GraphPad).

### Chrome Azurol S Plate Assay

Chrome azurol S (CAS) agar plates were prepared for the CAS assay. All components were purchased from Sigma except for agar, NaOH, NaCl (BioShop), Casamino Acids (Becton, Dickinson), and glucose (EMD Millipore). Stock solutions of TS, vancomycin (VAN), DSX, and DFP were standardized to 2 mg/ml. Five microliters of 2 mg/ml compound was spotted on a plate and incubated at room temperature for 1 h prior to photographing the plate.

### Generation of Efflux Mutants

Deletion mutants lacking the outer membrane components of the 4 major resistance-nodulation-division (RND) efflux systems of P. aeruginosa (MexAB-OprM, MexXY-OprM, MexCD-OprJ, and MexEF-OpmD) were generated. Briefly, the pairs of primers listed in Table 3 were used to amplify regions up- and downstream of the gene to be deleted. The PCR products were digested with the restriction enzymes indicated in the primer sequences and the resulting fragments ligated into the suicide vector pEX18Gm. After DNA sequencing validation of the constructs, they were introduced into E. coli SM10 for biparental mating into P. aeruginosa PAO1. Mating mixtures were plated on Pseudomonas isolation agar containing 200 μg/ml gentamicin (Gm) to counterselect the donor. Gm-sensitive double recombinants were selected on LB agar, with no salt and with 5% (wt/vol) sucrose. Gm-sensitive deletion mutants were identified by PCR and validated by DNA sequencing of the deletion junction.

TABLE 3
Primers used to generate
P. aeruginosa efflux mutants.

| | Sequence |
|---|---|
| Primer name | (restriction sites underlined) |
| Del-oprM-Eco-F1 | 5′-CGTTGGAATTCCTGGACCGGCCTGTCC |
| | TAC-3′ |
| Del-oprM-Sac-R1 | 5′-GGCCGGAGCTCCGCCGCGCCGGTGTTC |
| | TGC-3′ |
| Del-oprM-Sac-F2 | 5′-CGCTCGAGCTCGTTCACCGCGCAGCAG |
| | CAA-3′ |
| Del-oprM-Hind-R2 | 5′-CGGCCAAGCTTAATCGGCCGCCGGAAG |
| | TCG-3′ |
| Del-oprJ-Eco-F1 | 5′-GCCGGAATTCGGCTACGAGTGGACCGG |
| | CCT-3′ |
| Del-oprJ-Sac-R1 | 5′-CTCCGAGCTCGTCGGCCACCGGCGCGG |
| | CGG-3′ |
| Del-oprJ-Sac-F2 | 5′-CGATGAGCTCCGCAGCAGCTTCCTCAA |
| | CGA-3′ |
| Del-oprJ-Hind-R2 | 5′-TCTCGAAGCTTCAGCGCCAACCCCGTC |
| | GT-3′ |
| Del-opmD-Eco-F1 | 5′-GACTGGAATTCCGACTCGCGGCAATAC |
| | AC-3′ |
| Del-opmD-Sac-R1 | 5′-CGCTGGAGCTCGGGGCCGACGCTGCAG |
| | GC-3′ |
| Del-opmD-Sac-F2 | 5′-ACCAGAGCTCCGCGAGGAACTGGCGCA |
| | GGC-3′ |
| Del-opmD-Hind-R2 | 5′-CCTGAAGCTTGCTGCCCGGATGCCGGC |
| | CG-3′ |

### Results

Thiostrepton Stimulates P. aeruginosa Biofilm Formation

The inventors used a previously described P. aeruginosa biofilm assay (11) to screen a collection of ˜4000 bioactive molecules that includes 100 FDA-approved, off-patent drugs and antibiotics (12). The molecules were screened in duplicate at 10 μM in a dilute growth medium consisting of 10% lysogeny broth (LB), 90% phosphate buffered saline (henceforth, 10:90) to identify molecules capable of modulating biofilm formation. This medium was chosen to minimize the amount of biofilm formed in the presence of the vehicle control, so that molecules that stimulated biofilm formation could be more easily identified. The hits were divided into planktonic growth inhibitors (60 compounds), biofilm inhibitors (defined as those resulting in ≤50% of vehicle control biofilm, 8 compounds), or biofilm stimulators (those resulting in ≥200% of vehicle-treated control biofilm, 60 compounds) (Table 4). The hit rate ˜3% was relatively high for a primary screen, but all the molecules in this curated collection have biological activity. The hits belonged to a variety of chemical classes and included drugs with nominally eukaryotic targets.

TABLE 4
Compounds modulating Pseudomonas aeruginosa biofilm formation at 10 μM in 10:90 medium.

| | | | | |
|---|---|---|---|---|
| COMPOUND | VENDOR | CAT_NUM | FORMULA | MW |

GROWTH INHIBITORS

| | | | | |
|---|---|---|---|---|
| Coumermycin A1 | BIOMOL | GR-317 | C55H59N5O20 | 1110.08 |
| Patulin | BIOMOL | NP-223 | C7H6O4 | 154.12 |
| Harmane | BIOMOL | NP-122 | C12H10N2 | 182.22 |
| Echinomycin | BIOMOL | NP-090 | C51H64N12O12S2 | 1101.26 |
| 4-chloromercuribenzoic | LOPAC | C 5913 | C7H5C1HgO2 | 357.16 |
| acid | | | | |
| Diphenylenciodonium | LOPAC | D 2926 | C12H8C1I | 314.55 |
| chloride | | | | |
| Doxycycline hydrochloride | LOPAC | D 9891 | C22H25C1N2O8 | 480.90 |
| Demeclocycline | LOPAC | D 6140 | C21H22C12N2O8 | 501.31 |
| hydrochloride | | | | |
| Lomefloxacin | LOPAC | L 2906 | C17H20C1F2N3O3 | 387.81 |
| hydrochloride | | | | |
| Mitoxantrone | LOPAC | M 6545 | C22H30C12N4O6 | 517.40 |
| Ofloxacin | LOPAC | O 8757 | C18H20FN3O4 | 361.37 |
| Ruthenium red | LOPAC | R 2751 | C16H42N14O2Ru3 | 786.35 |
| Trimethoprim | LOPAC | T 7883 | C14H18N4O3 | 290.32 |
| Peucenin | MICROSOURCE | 100528 | C15H16O4 | 260.29 |
| Koparin | MICROSOURCE | 200422 | C16H12O6 | 300.26 |
| Pomiferin | MICROSOURCE | 201580 | C25H24O6 | 420.45 |
| Osajin | MICROSOURCE | 201595 | C25H24O5 | 404.46 |
| Theaflavin digallate | MICROSOURCE | 201515 | C43H32O20 | 868.70 |
| Purpurin | MICROSOURCE | 1505300 | C14H8O5 | 256.21 |
| Polymyxin b sulfate | MICROSOURCE | 1500492 | C56H100N16O17S | 1301.56 |
| Tetracycline hydrochloride | MICROSOURCE | 1500566 | C22H25C1N2O8 | 480.90 |
| Colistin sulfate | MICROSOURCE | 1505955 | C52H102N16O21S2 | 1351.59 |
| Pyrithione zinc | MICROSOURCE | 1500260 | C10H9N2O2S2Zn | 318.73 |
| Minocycline hydrochloride | MICROSOURCE | 1500414 | C23H28C1N3O7 | 493.94 |
| Oxytetracycline | MICROSOURCE | 1500457 | C22H25C1N2O9 | 496.90 |
| Sulfadiazine | MICROSOURCE | 1500546 | ClOH1ON4O2S | 250.28 |
| Thimerosal | MICROSOURCE | 1500572 | C9H9HgNaO2S | 404.81 |
| Phenylmercuric acetate | MICROSOURCE | 1500644 | C8H8HgO2 | 336.74 |
| Merbromin | MICROSOURCE | 1500637 | C20H8Br2HgNa2O6 | 750.65 |
| Meclocycline | MICROSOURCE | 1501118 | C29H27C1N2O14S | 695.05 |
| sulfosalicylate | | | | |
| Cetrimonium bromide | MICROSOURCE | 1503200 | C19H42BrN | 364.45 |
| Chloroxine | MICROSOURCE | 1503202 | C9H5C12NO | 214.05 |
| Clioquinol | MICROSOURCE | 1505114 | C9H5C1INO | 305.50 |
| Acrisorcin | MICROSOURCE | 1504218 | C25H28N2O2 | 388.50 |
| Gatifloxacin | MICROSOURCE | 1504272 | C19H22FN3O4 | 375.39 |
| Moxifloxacin | MICROSOURCE | 1504303 | C23H29C1FN3O4 | 465.95 |
| hydrochloride | | | | |
| Rifaximin | MICROSOURCE | 1505321 | C43H51N3O11 | 785.88 |
| Pefloxacine mesylate | MICROSOURCE | 1505305 | Cl8H24FN3O6S | 429.46 |
| Bismuth subsalicylate | MICROSOURCE | 1505412 | C7H5BiO4 | 362.09 |
| Sarafloxacin hydrochloride | MICROSOURCE | 1505314 | C20H18C1F2N3O3 | 421.83 |
| Gemifloxacin mesylate | MICROSOURCE | 1505802 | C19H24FN5O7S | 485.49 |
| Colistimethate sodium | MICROSOURCE | 1500206 | C57H103N16Na5O28S5 | 1735.79 |
| Alexidine hydrochloride | MICROSOURCE | 1503074 | C26H58C12N10 | 581.71 |
| Dibenzoylmethane | MICROSOURCE | 1505311 | C15H12O2 | 224.25 |
| Broxyquinoline | MICROSOURCE | 1500623 | C9H5Br2NO | 302.95 |
| Tetrachloroisophthalonitrile | MICROSOURCE | 1504101 | C8C14N2 | 265.91 |
| Auranofin | MICROSOURCE | 1505744 | C20H34AuO9PS | 678.48 |
| Dobutamine hydrochloride | MICROSOURCE | 1503212 | C18H24C1NO3 | 337.84 |
| Ciprofloxacin | PRESTWICK | Prestw-113 | C17H21C1FN3O4 | 385.82 |
| hydrochloride | | | | |
| Chlorhexidine | PRESTWICK | Prestw-143 | C22H30C12N10 | 505.45 |
| Aztreonam | PRESTWICK | Prestw-185 | C13H17N5O852 | 435.43 |
| Norfloxacin | PRESTWICK | Prestw-221 | Cl6H18FN3O3 | 319.33 |
| Minocycline hydrochloride | PRESTWICK | Prestw-315 | C23H28C1N3O7 | 493.94 |
| Cefoperazone dihydrate | PRESTWICK | Prestw-327 | C25H31N9O10S2 | 681.70 |
| Zaprinast | PRESTWICK | Prestw-335 | C13H13N5O2 | 271.27 |
| Amikacin hydrate | PRESTWICK | Prestw-395 | C22H47N5O15 | 621.63 |
| Piperacillin sodium salt | PRESTWICK | Prestw-755 | C23H26N5NaO7S | 539.54 |
| Merbromin | PRESTWICK | Prestw-787 | C20H14Br2HgNa2O9 | 804.70 |
| Azlocillin sodium salt | PRESTWICK | Prestw-821 | C20H22N5NaO6S | 483.47 |
| Doxycycline hyclate | PRESTWICK | Prestw-852 | C22H25C1N2O8 | 480.90 |

BIOFILM STIMULATORS (>200% DMSO CONTROL BIOFILM)

| | | | | |
|---|---|---|---|---|
| Geranylgeranoic acid | BIOMOL | AP-307 | C20H32O2 | 304.47 |
| Calmidazolium chloride | LOPAC | C 3930 | C31H23C17N2O | 687.70 |
| Daphnetin | LOPAC | D 5564 | C9H6O4 | 178.14 |
| 4-diphenylacetoxy-n-(2- | LOPAC | D-142 | C21H25C12NO2 | 394.34 |
| chloroethyl)piperidine | | | | |
| hydrochloride | | | | |
| S-(+)-fluoxetine | LOPAC | F 1553 | C17H19C1F3NO | 345.79 |
| hydrochloride | | | | |
| Fluphenazine | LOPAC | F 4765 | C22H28C12F3N3OS | 510.44 |
| dihydrochloride | | | | |
| 5-hydroxyindolacetic acid | LOPAC | H 8876 | Cl OH9NO3 | 191.18 |
| Loperamide hydrochloride | LOPAC | L 4762 | C29H34C12N2O2 | 513.50 |
| Maprotiline hydrochloride | LOPAC | M 9651 | C20H24C1N | 313.86 |
| Ci-976 (2,2-dimethyl-n- | LOPAC | C3743 | C23H39NO4 | 393.56 |
| (2,4,6- | | | | |
| trimethoxyphenyl)dodecanamide) | | | | |
| Niclosamide | LOPAC | N 3510 | C13H8C12N2O4 | 327.12 |
| Pimozide | LOPAC | P 1793 | C28H29F2N3O | 461.55 |
| Bay 11-7082 | LOPAC | B 5556 | Cl OH9NO2S | 207.25 |
| Tyrphostin a9 | LOPAC | T-182 | C18H22N2O | 282.38 |
| Khayanthone | MICROSOURCE | 100049 | C32H42O9 | 570.67 |
| Atranorin | MICROSOURCE | 200034 | Cl9H18O8 | 374.34 |
| Epicatechin monogallate | MICROSOURCE | 210238 | C22H18O10 | 442.37 |
| Retusin 7-methyl ether | MICROSOURCE | 240645 | C17H14O5 | 298.29 |
| Larixol | MICROSOURCE | 300056 | C20H34O2 | 306.48 |
| Chrysanthemic acid, ethyl | MICROSOURCE | 310019 | C12H20O2 | 196.29 |
| ester | | | | |
| Larixinic acid | MICROSOURCE | 310025 | C6H6O3 | 126.11 |
| Chaulmoogric acid | MICROSOURCE | 310016 | C18H32O2 | 280.45 |
| Harmol hydrochloride | MICROSOURCE | 1502237 | C12H11C1N2O | 234.68 |
| Hederagenin | MICROSOURCE | 1504016 | C30H48O4 | 472.70 |
| Cytisine | MICROSOURCE | 1504027 | C11H14N2O | 190.24 |
| Fraxetin | MICROSOURCE | 1504069 | C10H8O5 | 208.17 |
| Ursolic acid | MICROSOURCE | 1800031 | C30H48O3 | 456.70 |
| Diallyl sulfide | MICROSOURCE | 1505293 | C6H1 OS | 114.21 |
| Deferoxamine mesylate | MICROSOURCE | 1500224 | C26H52N6O11S | 656.79 |
| Chlortetracycline | MICROSOURCE | 1500186 | C22H24C12N2O8 | 515.34 |
| hydrochloride | | | | |
| Hexachlorophene | MICROSOURCE | 1500328 | Cl3H6C16O2 | 406.90 |
| Gramicidin | MICROSOURCE | 1500319 | C60H92N12O10 | 1141.45 |
| Mechlorethamine | MICROSOURCE | 1500375 | C5H11C12N | 156.05 |
| Piperacillin sodium | MICROSOURCE | 1500489 | C23H26N5NaO7S | 539.54 |
| Sulfapyridine | MICROSOURCE | 1500551 | C11H11N3O2S | 249.29 |
| Sulfathiazole | MICROSOURCE | 1500553 | C9H9N3O2S2 | 255.32 |
| Thiamphenicol | MICROSOURCE | 1503136 | Cl2H15C12NO5S | 356.22 |
| Azithromycin | MICROSOURCE | 1503679 | C38H72N2O12 | 748.98 |
| Thiram | MICROSOURCE | 1503322 | C6H12N2S4 | 240.43 |
| Dirithromycin | MICROSOURCE | 1504144 | C42H78N2O14 | 835.07 |
| Telithromycin | MICROSOURCE | 1505265 | C43H65N5O10 | 812.00 |
| Thiostrepton | MICROSOURCE | 1505111 | C72H85N19O18S5 | 1664.89 |
| Triclosan | MICROSOURCE | 1505465 | Cl2H7C13O2 | 289.54 |
| Trientine hydrochloride | MICROSOURCE | 1505675 | C6H20C12N4 | 219.16 |
| Bromperidol | MICROSOURCE | 1505972 | C21H23BrFNO2 | 420.32 |
| Lipoamide | MICROSOURCE | 1505740 | C8H15NOS2 | 205.34 |
| Aliskiren hemifumarate | MICROSOURCE | 1505710 | C34H57N3O10 | 667.83 |
| Chloramphenicol | MICROSOURCE | 1500173 | Cl5H16C12N2O8 | 423.20 |
| hemisuccinate | | | | |
| Thioctic acid | MICROSOURCE | 1503941 | C8H14O2S2 | 206.33 |
| Pipemidic acid | MICROSOURCE | 1502024 | C14H17N5O3 | 303.32 |
| Carmofur | MICROSOURCE | 1505317 | Cl1H16FN3O3 | 257.26 |
| Hygromycin b | MICROSOURCE | 1505362 | C20H37N3O13 | 527.52 |
| Dichlorophene | MICROSOURCE | 1500626 | Cl3H10C12O2 | 269.12 |
| Rosmarinic acid | MICROSOURCE | 1502094 | C18H16O8 | 360.31 |
| Dropropizine | MICROSOURCE | 1501004 | Cl3H2ON2O2 | 236.31 |
| 2,4-dichlorophenoxyacetic | MICROSOURCE | 330048 | Cl6H22C12O3 | 333.25 |
| acid, isooctyl ester | | | | |
| Atracurium besylate | PRESTWICK | Prestw-5 | C65H82N2O18S2 | 1243.48 |
| Chloramphenicol | PRESTWICK | Prestw-31 | Cl1H12C12N2O5 | 323.13 |
| Dihydrostreptomycin | PRESTWICK | Prestw-159 | C42H88N14O36S3 | 1461.42 |
| sulfate | | | | |
| Cefotetan | PRESTWICK | Pre stw-473 | Cl7H17N7O8S4 | 575.62 |
| Dacarbazine | PRESTWICK | Pre stw-574 | C6H1ON6O | 182.18 |
| Roxithromycin | PRESTWICK | Prestw-854 | C41H76N2O15 | 837.05 |
| Thonzonium bromide | PRESTWICK | Prestw-925 | C32H55BrN4O | 591.71 |
| (soap) | | | | |
| Flucytosine | PRESTWICK | Prestw-934 | C4H4FN3O | 129.09 |
| Florfenicol | PRESTWICK | Prestw-955 | Cl2H14C12FNO4S | 358.21 |
| Apramycin | PRESTWICK | Pre stw-1005 | C21H41N5O11 | 539.58 |
| Ramipril | PRESTWICK | Prestw-1107 | C23H32N2O5 | 416.51 |
| Cefepime hydrochloride | PRESTWICK | Prestw-1118 | C19H27C1N6O6S2 | 535.04 |

BIOFILM INHIBITORS (<50% DMSO CONTROL BIOFILM)

| | | | | |
|---|---|---|---|---|
| L-3,4- | LOPAC | D 1507 | C10H14C1NO4 | 247.68 |
| dihydroxyphenylalanine | | | | |
| methyl ester hydrochloride | | | | |
| R(-)-propylnorapomorphine | LOPAC | D-027 | C19H22C1NO2 | 331.84 |
| hydrochloride | | | | |
| Nordihydroguaiaretic acid | LOPAC | N 5023 | C18H22O4 | 302.36 |
| from Larrea divaricata | | | | |
| (creosote bush) | | | | |
| N-oleoyldopamine | LOPAC | O 2139 | C26H43NO3 | 417.62 |
| Curcumin | MICROSOURCE | 1505345 | C21H20O6 | 368.38 |
| 3,5-dihydroxyflavone | MICROSOURCE | 1505147 | C15H10O4 | 254.24 |
| 3-hydroxy-3′,4′- | MICROSOURCE | 1505278 | C17H14O5 | 298.29 |
| dimethoxyflavone | | | | |
| Limonin | MICROSOURCE | 1800018 | C26H30O8 | 470.51 |

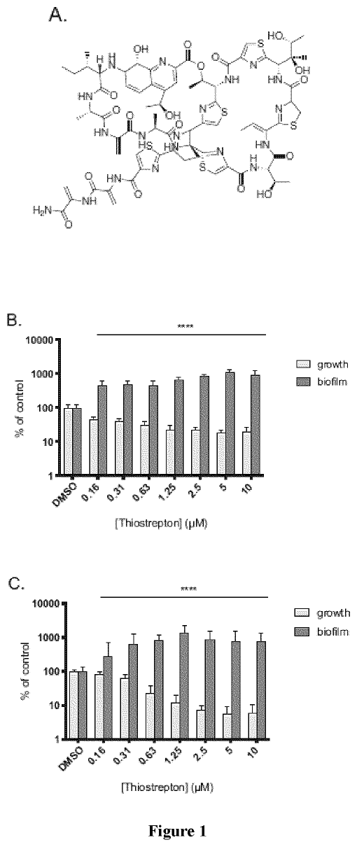

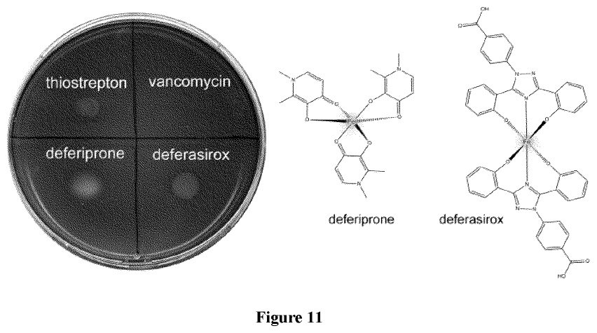

Among the molecules in the screen that stimulated biofilm formation was the thiopeptide antibiotic, thiostrepton (TS; FIG. 1A). This response was intriguing because TS—a form of bacteriocin—is considered ineffective against Gram-negative bacteria due to the impermeability of the outer membrane (GM) to large hydrophobic compounds (13, 14). In dose-response experiments in 10:90 medium, biofilm increased while planktonic cell density decreased with increasing TS concentrations to 10 μM (17 μg/ml), the maximum that could be tested due to its limited solubility (FIG. 11B).

Growth in Minimal Media Increases Susceptibility of P. aeruginosa to TS

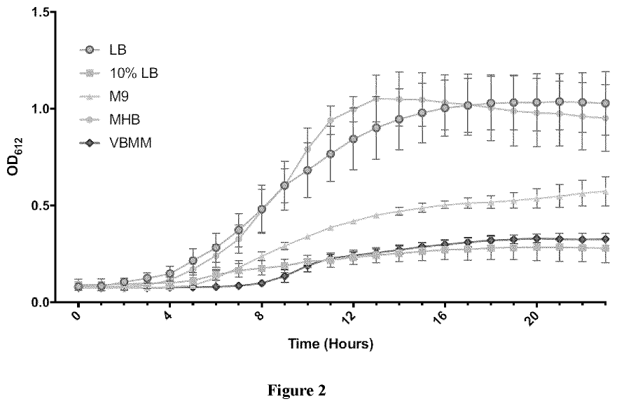

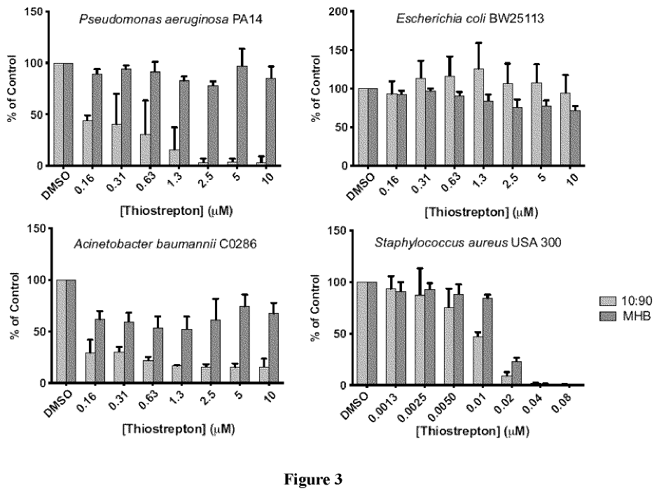

Environmental conditions can modulate the expression or essentiality of antibiotic targets or alter the availability of particular nutrients, leading to changes in susceptibility. It was hypothesized that the biofilm response of P. aeruginosa to TS may be the result of nutrient deficiency in 10:90, which was more limiting to P. aeruginosa growth than M9 minimal medium (FIG. 2). Growth rates in Vogel Bonner Minimal Media (VBMM) in the absence of TS was similar to that in 10:90 (FIG. 2) but in the presence of TS, planktonic cell density decreased to below the level of detection at concentrations above ˜0.63 μM (FIG. 1C). Consistent with this idea, the growth of P. aeruginosa in nutrient-rich Mueller-Hinton broth (MHB) reduced susceptibility to TS (FIG. 3). These data suggested that nutrient limitation enhances susceptibility of P. aeruginosa to TS.

The Ribosomal Methyltransferase Tsr Protects P. aeruginosa Against TS

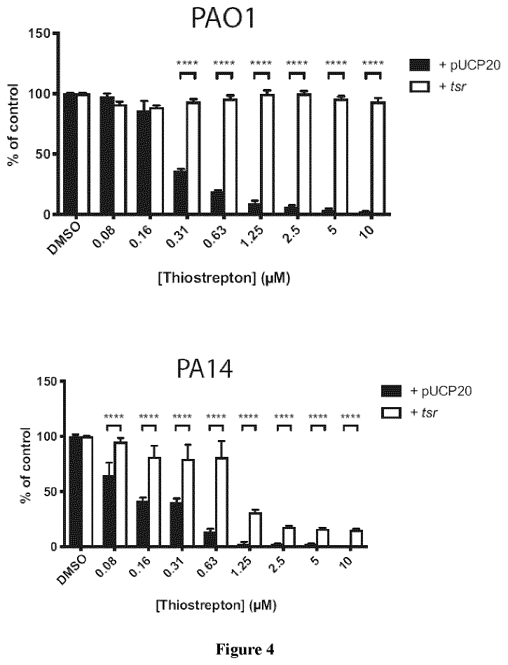

The established MOA for TS antibacterial activity is inhibition of protein translation through direct binding to bacterial ribosomes. However, because TS has broad anti-parasitic and anti-neoplastic activities, the possibility that it might inhibit P. aeruginosa growth in a novel way was considered. To validate the MOA, a resistance gene was expressed, tsr, from a plasmid in P. aeruginosa strains PAO1 and PA14. tsr encodes a 23s rRNA methyltransferase, used by TS producer Streptomyces azureus to prevent self-intoxication. Tsr methylates the conserved A1067 residue of 23s rRNA, impairing binding of TS to its target. Expression of tsr in trans increased resistance of both PAO1 and PA14 compared to vector-only controls (FIG. 4). PAO1 was resistant up to the maximum soluble TS concentration of 10 μM, while resistance of PA14 was significantly increased compared to control, although not to the same extent as PAO1. These results suggest that TS inhibits growth via its canonical MOA of ribosome binding, implying that it can cross the P. aeruginosa OM to access the bacterial cytoplasm.

### TS Susceptibility Increases in the Presence of Iron Chelators

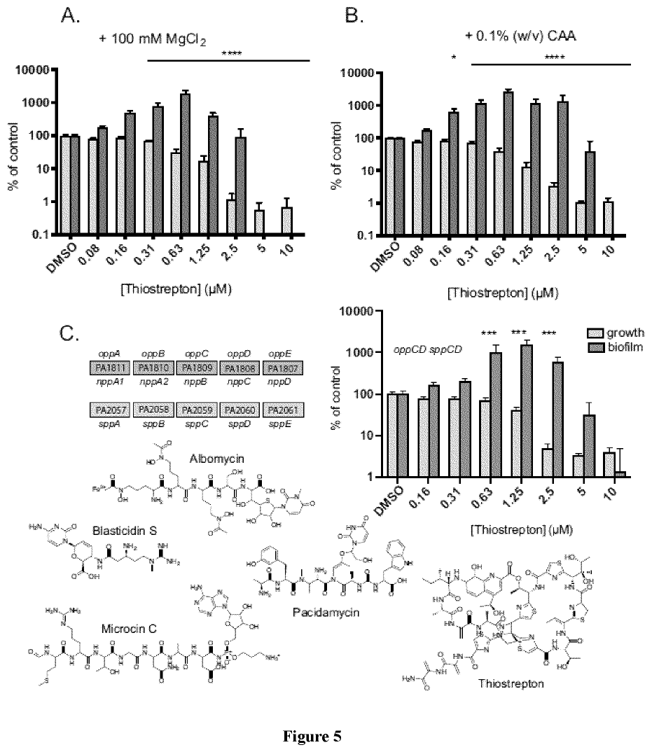

To understand the reason for increased TS susceptibility of P. aeruginosa in VBMM compared to 10:90 medium, differences in nutrient availability between the two media types was considered. The primary carbon source in 10% LB is amino acids while the carbon source in VBMM is citrate. Citrate can chelate divalent cations including calcium and magnesium, which are important for OM integrity. It was hypothesized that this chelation effect may increase OM permeability. To stabilize the OM, the dose response assay was repeated in VBMM supplemented with 100 mM MgCl₂ but no effect on susceptibility was observed (FIG. 5A). Since TS is a thiopeptide, it was hypothesized that amino acid limitation during growth in VBMM may increase uptake of TS, leading to growth inhibition. To test this, VBMM supplemented with 0.1% casamino acids, but saw no change in TS susceptibility (FIG. 5B). Further, simultaneous deletion of components of the Opp (Npp) peptide transport system, exploited by other peptide antibiotics for entry, and a homologous system, Spp, had no effect on TS susceptibility (FIG. 5C).

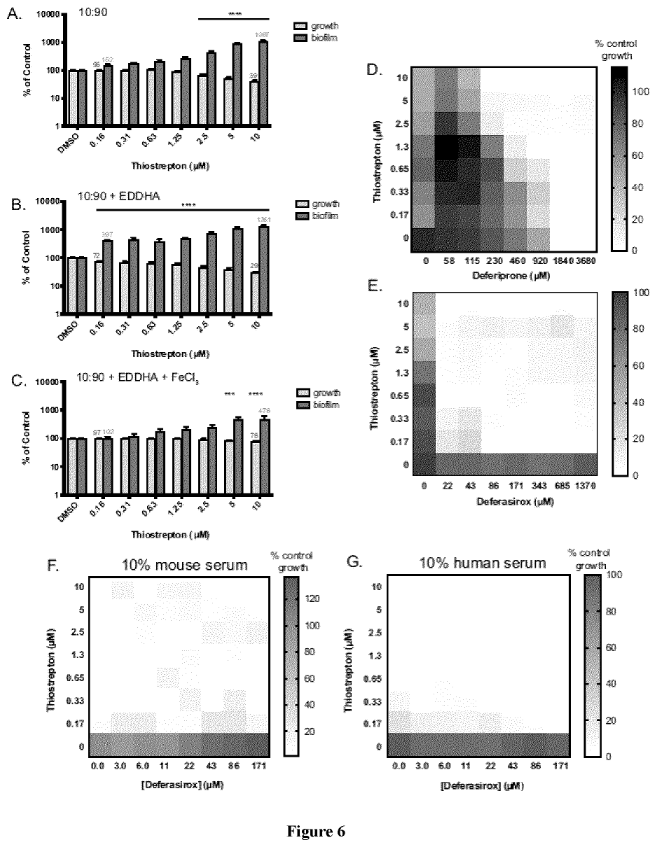

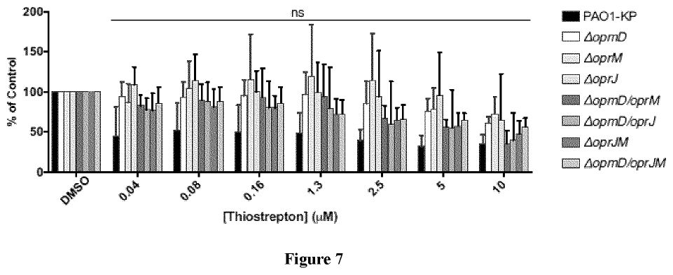

The inventors next considered that VBMM was more iron-limited than 10:90 medium, which contains trace iron from yeast extract and peptone. Under iron limitation, bacteria secrete siderophores into the extracellular milieu to scavenge iron. Specialized receptors then transport siderophore-iron complexes back into the cell. Some antibiotics, including sideromycins, pyocins and bacteriocins, use siderophore receptors to access intracellular targets, and it was hypothesized that TS may use this strategy. The inventors compared P. aeruginosa PAO1 grown in 10:90 with increasing concentrations of TS alone (FIG. 6A) or with 0.1 μM EDDHA, a membrane-impermeable iron chelator (FIG. 6B). Addition of EDDHA shifted biofilm stimulation and growth inhibition to lower concentrations of TS compared to 10:90 alone. Supplementation of 10:90 plus 0.1 μM EDDHA with 100 μM FeCl₃ increased planktonic cell density and reduced biofilm stimulation (FIG. 6C). These data suggest that TS susceptibility is inversely proportional to iron availability, and that TS may exploit siderophore receptors to cross the OM of P. aeruginosa. Consistent with the active import of this antibiotic, mutants lacking the major efflux pumps of P. aeruginosa had near-wild-type susceptibility to TS (FIG. 7).

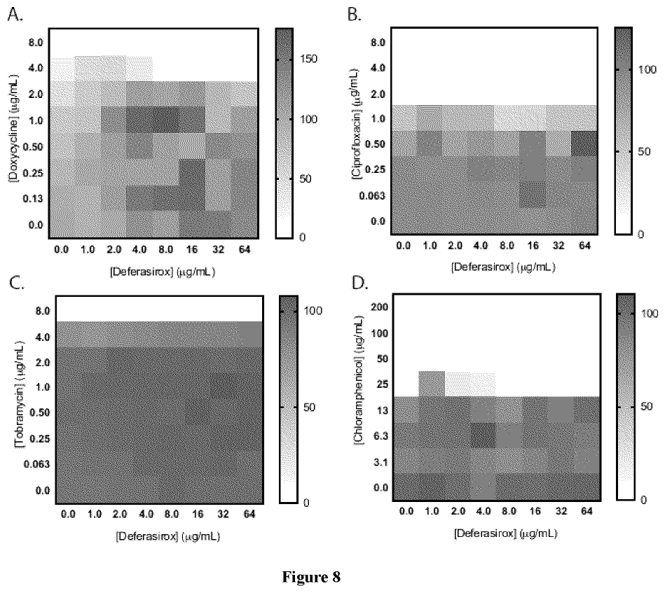

The poor solubility of TS has hampered its development as a therapeutic, but these data suggested that its effective concentration could be reduced in the presence of iron chelators. The FDA-approved iron chelators deferiprone (DFP) and deferasirox (DSX) were tested for potential synergy with TS. Checkerboard assays revealed that while neither chelator had activity against P. aeruginosa on its own, both potentiated TS activity (FIGS. 6D and 6E) at concentrations well below those used to safely treat patients, up to 28 mg/kg/day for DSX or 99 mg/kg/day for DFP. These effects are specific for TS, as DSX failed to synergize with other tested antibiotics that do not depend on iron availability for uptake (FIG. 8).

High-affinity iron chelation by transferrin, hemoglobin, and lactoferrin is a common strategy used by mammals to restrict the growth of microorganisms. It was investigated whether serum could also potentiate TS activity. Interestingly, the addition of 10% heat-inactivated mouse or human serum to 10:90 medium markedly decreased the concentration of TS required to inhibit growth, regardless of the presence of DSX (FIGS. 6F and 6G), suggesting that the levels of iron were already very low under these conditions.

### TS Hijacks Pyoverdine Receptors FpvA and FpvB

To identify the route of iron-limitation dependent TS entry into P. aeruginosa, the susceptibility of mutants from the ordered PA14 transposon library (15) that had insertions in genes encoding known siderophore receptors, as well as mutants with insertions in uncharacterized OM proteins with homology to siderophore receptors was tested. In VBMM, most mutants had MICs similar to those of the parental strain (Table 1). In contrast, an fpvA mutant, encoding the type I pyoverdine receptor, had an MIC of 7.5 μM. Growth inhibition was still observed at the highest TS concentrations, indicating that the fpvA mutant remained partially susceptible. P. aeruginosa encodes two type I pyoverdine receptors, FpvA and FpvB, with ˜39% amino acid identity (71% similarity). The fpvB mutant was also less susceptible to TS than the parent strain, with an MIC of 1.3 μM. Based on these patterns of susceptibility, it was speculated that TS may use both FpvA and FpvB, but that FpvA was the preferred receptor. When fpvA was deleted in the fpvB background, the double mutant had similar resistance levels to the fpvA single mutant (Table 1), but complementation of that mutant with fpvB on a low-copy number plasmid increased TS susceptibility (Table 1). Together, these data suggest that TS exploits both pyoverdine receptors for entry.

### TS is Active Against Multi-Drug Resistant Clinical Isolates

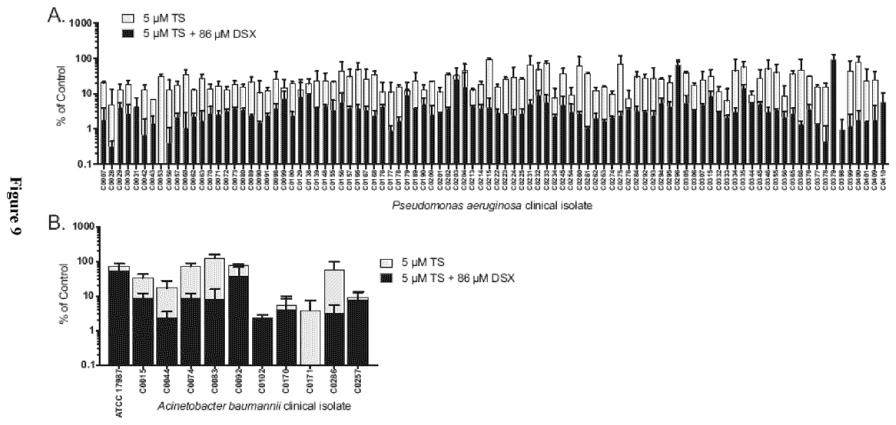

To test whether TS could inhibit growth of a broader range of P. aeruginosa strains, particularly multi-drug resistant (MDR) isolates for which there are fewer antibiotic options, 96 recent clinical isolates were tested for susceptibility to TS in 10:90 medium. While approximately 1 in 10 of those strains had an MIC≥5 μM TS (FIG. 9A), a combination of 5 μM TS (8.3 μg/ml) plus 86 μM DSX (32 μg/ml) reduced growth of all but three isolates of P. aeruginosa to less than 20% of the DMSO control (FIG. 9A). The activity of TS was next tested against another MDR Gram-negative pathogen that can cause severe infections, Acinetobacter baumanii. A. baumannii strains encode FpvA and FpvB homologs (FIG. 5), suggesting they may be susceptible to the thiopeptide. Growth of 6 of 10 A. baumannii strains in 10:90 was reduced to ≤50% of control with 5 μM TS, while the combination of 5 μM TS and 86 μM DSX reduced growth of 8/9 clinical isolates of A. baumanii below 20% of control (FIG. 9B). The growth of Escherichia coli (which lacks FpvAB homologs) was unaffected in 10:90 medium even at the maximum soluble concentration of 10 μM (17 μg/ml) TS (FIG. 3). The growth of methicillin-resistant Staphylococcus aureus USA300 was inhibited by <40 nM (32 to 64 ng/ml) of TS in both 10:90 medium and MHB, showing that iron limitation has little effect on TS susceptibility in the absence of an outer membrane.

### Discussion

The natural role of antibiotics has been broadly debated, prompting questions such as, are they signaling molecules that are toxic at high concentrations, or weapons used by bacteria to gain an advantage over competitors in their environment? The biofilm stimulation response to sub-inhibitory concentrations of antibiotics is consistent with both views. At concentrations too low to elicit damage, bacteria show little phenotypic response to antibiotic exposure. As concentrations approach the MIC, the bacteria respond in a dose-dependent manner by ramping up the amount of biofilm produced—detecting either the antibiotics or their effects on the cell—which may protect a subpopulation of cells. Above the MIC, antibiotics fall into the deadly weapons category. Biofilm stimulation by sub-inhibitory concentrations of antibiotics is a common phenomenon, reported for multiple gram-positive and gram-negative species, and for several drug classes, suggesting that it is not linked to a specific MOA. As demonstrated here, this phenomenon can be used to identify potential antibiotic activity in the absence of killing, a useful feature when screening at a single concentration that may be below the MIC for the drug-organism combination being used. Interestingly, it has been found (16) that many drugs intended for eukaryotic targets can impact bacterial growth and biofilm formation (Table 4), implying that they have deleterious effects on prokaryotic physiology. With a new appreciation of the role of the human microbiome in health and disease, these potential effects should be considered during drug development.

TS, a complex cyclic thiopeptide made by Streptomyces azureus, S. hawaiiensis, and S. laurantii, is experiencing a resurgence of research interest due to its broad anti-bacterial, anti-malarial, and anti-cancer activities (24, 25). It is a member of the RiPP (ribosomally synthesized and post-translationally modified peptides) class of natural products (41), derived from a 42-amino acid precursor, TsrA (19). Although the mechanism of its antibacterial activity (inhibition of translation by binding to helices H43/H44 of 23S rRNA) and resistance (methylation of 23S rRNA residue A1067) have been deciphered (27, 42), the way in which this ˜1.7 kDa molecule enters target bacteria is unknown. These data suggest that TS is actively imported into P. aeruginosa under iron-restricted conditions. Its large mass would impede passive diffusion through the outer membrane, and single, double, or triple mutants lacking the outer membrane components of major efflux systems MexAB-OprM, MexCD-OprJ, and MexEF-OpmD have wild-type TS susceptibility.

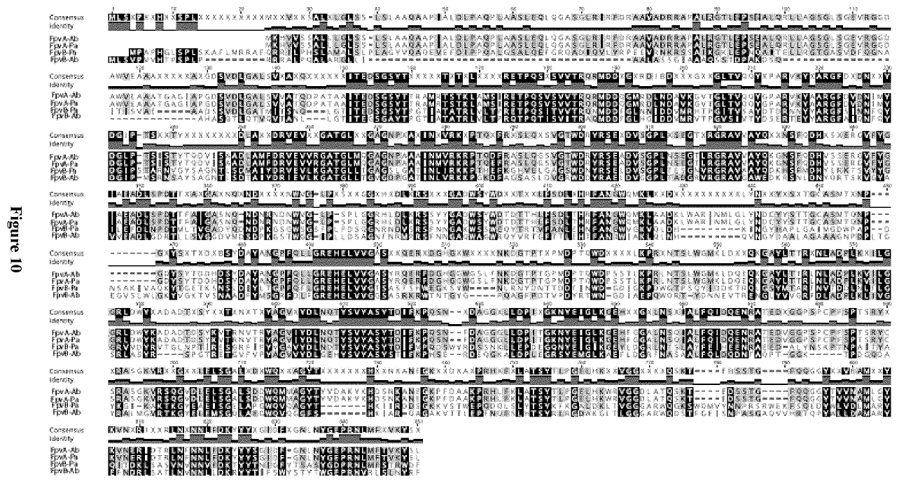

There are multiple examples of molecules that exploit iron uptake pathways to enter bacteria. Class I microcins—narrow-spectrum antibiotics produced by some gram negative species—bind to siderophore receptors and share many of TS's properties. They are RiPPs, less than 5 kDa in mass, and cyclic (giving them the nickname ‘lasso peptides’). Notably, binding of iron by microcins is not a prerequisite for uptake, as some interact with siderophore receptors in an iron-free state. For example, MccJ25, produced by E. coli, interacts with siderophore receptor FhuA by mimicking the structure of ferrichrome. Although TS has multiple hydroxyls positioned in a manner that could potentially coordinate metals (FIG. 1A), it is unlikely to bind iron based on its inability to decolorize chrome azurol S agar, which changes from blue to orange/yellow when iron is bound by a ligand, in comparison to chelators DFP and DSX (FIG. 10). Further, the structure of TS has been solved both by X-ray crystallography and nuclear magnetic resonance (NMR), and no bound metals were reported (17, 18).

The discovery that TS exploits pyoverdine receptors FpvA and FpvB for uptake into the periplasm helps to explain the resistance of gram-negative species such as E. coli to this antibiotic, as they lack those proteins. FpvAB homologs are expressed by P. aeruginosa and related bacteria—including A. baumanii (FIG. 5)—suggesting that TS could have utility across different species that express pyoverdine receptors. FpvA is also exploited by S-pyocins, 40- to 80-kDa peptide antibiotics produced by competing P. aeruginosa strains, showing that it is an important promiscuous access point for diverse molecules in addition to its pyoverdine ligand. Use of multiple pyoverdine receptors by TS may reduce the probability of resistance arising through mutation of a single receptor, although genome analysis of clinical isolate C0379 that was most resistant to the combination of TS and DSX (FIG. 9A) revealed a wild type copy of fpvA coupled with an ˜800 bp deletion encompassing the 5′ region of fpvB. Multiple single-nucleotide polymorphisms with unknown effects on function were present in both TS-susceptible and -resistant isolates.

Although TS uses siderophore receptors to cross the P. aeruginosa OM, the way in which this large cyclic peptide transits the cytoplasmic membranes of gram-positive and gram-negative bacteria to reach its ribosomal targets remains undefined. Expression of tsr in P. aeruginosa conferred resistance, confirming that TS acts at least in part via its canonical bacteriostatic MOA. While PA14 expressing Tsr was significantly more resistant to TS than the control, it was more sensitive than PAO1. This difference is not due to nucleotide polymorphism at the Tsr methylation site on the rRNA, as these residues are conserved between PAO1 and PA14. The reasons for strain-specific differences in susceptibility are unclear, but these data confirm that most P. aeruginosa isolates tested (including MDR strains) are susceptible to TS, especially when it is combined with DSX (FIG. 9A).

The major liability of TS is its poor solubility. Smaller, more soluble fragments that retain activity against gram-positive bacteria and have reduced toxicity for eukaryotic cells have been identified but it is not clear if they would be active against P. aeruginosa or A. baumannii if uptake by FpvAB requires the intact molecule. Another way to manage solubility issues is to reduce the required concentration required to kill. These data show that this can be accomplished for TS by co-administration with FDA-approved iron chelators DFP or DSX (FIGS. 6D and 6E). The true potential of TS as an anti-infective may be underestimated, as MIC evaluations are typically performed in rich, iron-replete media such as cation-adjusted Mueller-Hinton broth. Many host environments are iron-restricted, particularly in the presence of infection and inflammation. Our data show that TS is active at low-micromolar concentrations against P. aeruginosa in 10% mouse and human sera, even in the absence of added chelator. The combination of TS with DSX may be useful at sites such as chronically infected lungs, where iron is more abundant.

In summary, it was shown that biofilm stimulation can be used in high throughput small molecule screening to report on sub-inhibitory antibiotic activity that may otherwise be missed using the conventional criterion of growth inhibition. In a small screen of less than 4000 molecules at a fixed concentration of 10 μM, 60 molecules were identified that stimulated biofilm formation, suggesting that they may have antimicrobial activity at higher concentrations, or under slightly different growth conditions, as demonstrated here for TS. Stimulation of biofilm matrix production by TS in the gram-positive genus Bacillus was reported previously, and that phenotype leveraged to identify novel thiopeptide producers in co-cultures (19). Those studies, and the data presented here, suggest that monitoring biofilm stimulation (or an easily assayed proxy thereof, such as increased expression from biofilm matrix promoters) could allow for more sensitive detection of molecules with potential antibacterial activity during screening, making it a useful addition to the antimicrobial discovery toolkit.

### Example 2. A Combination of Thiostrepton and One or More Iron-Binding Compounds are Effective in Inhibiting P. aeruginosa and Acinetobacter baumanii

The small number of P. aeruginosa and A. baumannii strains resistant to TS-chelator combinations, prompted the inventors to look for new compounds that could synergize with TS to inhibit those clinical isolates. From literature surveys 14 compounds reported to have iron-chelating activity, plus one iron analogue, were selected and tested for synergy with TS. Doxycycline (DOXY), ciclopirox olamine (CO), tropolone (TRO), clioquinol (CLI), and gallium nitrate (GN) synergized with TS. Individual compounds were bacteriostatic but the combinations were bactericidal. Spectrophotometric data and chrome azurol S agar assay confirmed that the chelators potentate TS activity through iron sequestration rather than through their innate antimicrobial activities. A triple combination of TS+DSX+DOXY had the most potent activity against P. aeruginosa and A. baumannii isolates. Growth of one highly-resistant P. aeruginosa clinical isolate was inhibited with higher concentrations of three of the compounds in combination with TS+DSX.

### Methods

### Compounds

Compounds from Table 5 were from AK Scientific, Sigma, and Cayman Chemicals. TS and DSX were from Cayman Chemicals and AK Scientific respectively. Compounds were stored at −20° C. Stock solutions were stored at −20° C. until use except for the tetracyclines, which were made fresh due to precipitation at −20° C.

TABLE 5
The structures of literature-derived compounds tested (with potential iron chelation sites bolded).

| Compound | Structure | Description |
|---|---|---|
| Baicalin | 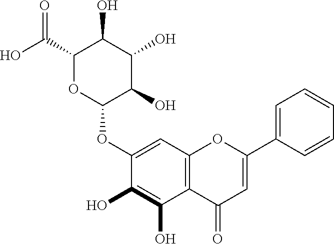 | Flavonoid isolated from the Chinese herb Scutellaria baicalensis with antioxidant, anti-inflammatory and anticancer activity. |
| Ferulic Acid | 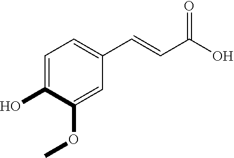 | A natural product found in plant cell walls with antioxidant activity. |
| Sodium phytate | 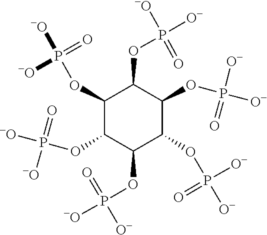 | A naturally occurring compound found in wheat and rice with anticancer and antioxidant activity. |
| 2,3,5,6- Tetramethylpyrazine | 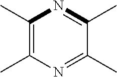 | An alkaloid derived from the Chinese herb Ligusticum wallichii that is used to treat vascular diseases. |
| Curcumin | 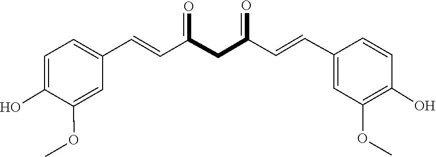 | Natural product of turmeric with anticancer activity. |
| Epigallocatechin Gallate | 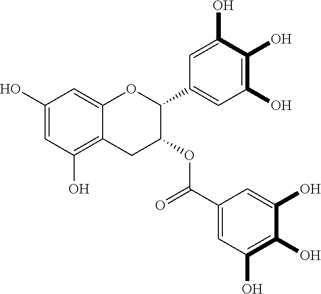 | A polyphenol isolated from green tea extract. |
| Tropolone | 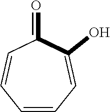 | Synthetic compound with broad- spectrum antimicrobial activity. |
| Clioquinol | 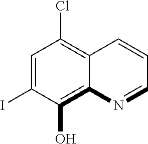 | Used to treat fungal and Bacterial infections. Also used in the treatment of Alzheimer's Disease. |
| Gallium Nitrate | 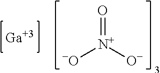 | Iron analogue with antimicrobial activity. |
| Ciclopirox Olamine | 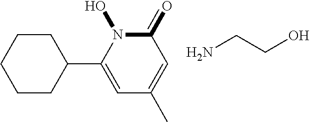 | Antifungal agent. |
| Phloretin | 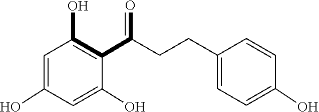 | A flavonoid found in apples and pears. |
| Apocynin | 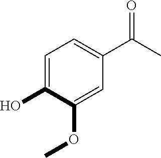 | An NADPH-oxidase Inhibitor. |
| Dexrazoxane | 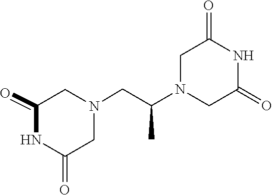 | Cardioprotective agent. |
| Eltrombopag | 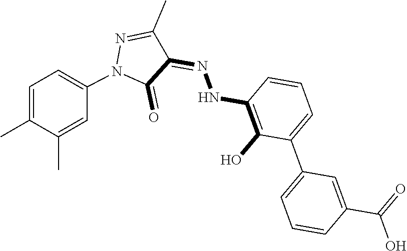 | A thrombopoietin receptor agonist used to treat thrombocytopenia. |
| Lipofermata | 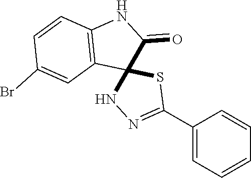 [SMILES: O=C1Nc2ccc(Br)cc2[C@@]12NN=C(c1ccccc1)S2] | Fatty acid transport inhibitor. |

### Absorption Spectra Assays for Iron Chelation

Compounds were arrayed in Nunc 96 microwell plates. Vehicle controls contained Milli-Q H₂O with 1:75 dilution of each compound at a final concentration of 300 Wi. The experimental set-up contained the same components as the vehicle control, with the addition of 300 Wi FeCl₃. The final volume in each well was 150 μL. The plate was incubated at room temperature for one hour and absorption spectra from 300 nm to 700 nm was read in 2 nm increments (Multiskan Go—Thermo Fisher Scientific). For the qualitative assay to identify potential antibiotic-Fe³⁺ Complexes, the concentrations of each antibiotic stock were: TOB (tobramycin 4 mg/mL), GENT (gentamicin 10 mg/mL), DSX (deferasirox 20 mg/mL), DFP (deferiprone 60 mg/mL), OFL (ofloxacin 4 mg/mL), PPA (pipemedic acid 64 mg/mL), CIP (ciprofloxacin 5 mg/mL), DOXY (doxycycline 50 mg/mL), TET (tetracycline 20 mg/mL), MINJ (minocycline 20 mg/mL), CAR (carbenicillin 100 mg/mL), PIP (piperacillin 6 mg/mL), CEFU (cefuroxime 30 mg/mL), CEFO (cefotaxime 30 mg/mL), AMP (ampicillin 30 mg/mL), CLOX (cloxacillin 30 mg/mL), MEC (mecillinam 30 mg/mL), CEFT (ceftriaxone 30 mg/mL), CEFIX (cefixime 12 mg/mL), VAN (vancomycin 30 mg/mL), TS (thiostrepton 20 mg/mL), CHLOR (chloramphenicol 50 mg/mL), POLY (polymyxin B 4 mg/mL), TRI (trimethoprim 50 mg/mL).

### CAS Assay

CAS agar plates were prepared as described in Example 1. Compounds were standardized to 2 mg/mL and 10 μL of each was spotted onto the plate. Plates were incubated at room temperature for 1 h, then photographed. Three replicates were conducted and the image of a representative plate was presented.

### Dose Response and Checkerboard Assays

Culture conditions, growth assays, dose response and checkerboard assays using P. aeruginosa PA14 were performed as described in Example 1. Briefly, overnight cultures were grown in LB for 16 h, 37° C., 200 rpm then subcultured (1:500 dilution) into 10:90 for 6 h. Subcultures were standardized to OD₆₀₀ of 0.10 and diluted 1:500 in fresh 10:90 before use. For the dose response assay, serial dilutions of compounds were added at 75 times the final concentration and diluted with 10:90 with cells to reach the desired final concentration. This was done in triplicate for technical replicates. Vehicle and sterile controls were included. The checkerboard assay was done similarly to the dose response assay but in an 8×8 format in a 96-well Nunc plate, with concentration of one drug increasing along the y-axis and the other along the x-axis. Sterility and vehicle controls were included with two columns allocated for each control. At least three biological replicates were repeated for the dose response and checkerboard assays.

### 3D Checkerboard Assays

Three-dimensional checkerboard assays were performed in Nunc 96 microwell plates in an 8×8×8 matrix format for a total of 512 wells. The first two columns were used for the vehicle controls while the last two columns were allocated to sterility controls, both consisting of 2.7% (v/v) DMSO+1.3% (v/v) H₂O for plates with TRO and DOXY and 4% (v/v) DMSO for plates with CLI and CO. Serial dilutions of TS were added along the y-axis of each plate starting at 0 μg/mL, with the highest final concentration being 4 μg/mL. Serial dilutions of DSX were added along the x-axis of each plate, from 0 μg/mL to the highest final concentration of 8 μg/mL. Serial dilutions of compound were added with an increasing concentration in each plate up to a final concentration of 35 μg/mL (TRO), 8 μg/mL (DOXY), 30 μg/mL (CO), and 8 μg/mL (CLI) in the last plate. Each well contained 144 μL of 10:90 inoculated with PA14, except for the sterility control columns which contained 10:90 only. The final volume in each plate was 150 μL. The plates were sealed with parafilm and incubated at 37° C. for 16 h, shaking at 200 rpm. The OD₆₀₀ of the plates was read (Multiskan Go—Thermo Fisher Scientific). Each experiment was repeated at least three times. Checkerboards were analyzed in Excel. Representative plots at % MIC were made using MATLAB. Surface areas were averaged, expressed in % of control, and plotted against each compound concentration (Prism, Graphpad).

### Clinical Isolate Testing

Clinical isolates were grown and tested as described in Example 1. Briefly, clinical isolates were inoculated from glycerol stocks stored at −80° C. into Nunc 96-well plates and grown overnight at 37° C., for 16 h with shaking in LB (200 rpm). Overnights were subcultured (1:25) into fresh 10:90 medium and grown for 2 h under the same growth conditions. Subcultures were diluted 1:75 in fresh 10:90. Compounds were diluted 1:75 to obtain the final concentration. DOXY and CLI were added at a final concentration of 1 μg/mL, CO was used at 2 μg/mL, TRO was used at 4 μg/mL, TS was used at 8.3 μg/mL, and DSX was used at 32 μg/mL. Vehicle and sterility controls were included. Plates were incubated overnight with the same conditions. The OD₆₀₀ was read (Multiscan Go—Thermo Fisher Scientific), analyzed using Excel, and the data plotted using Prism (GraphPad).

### Results

### Iron-Binding Antibiotics Form Coloured Complexes

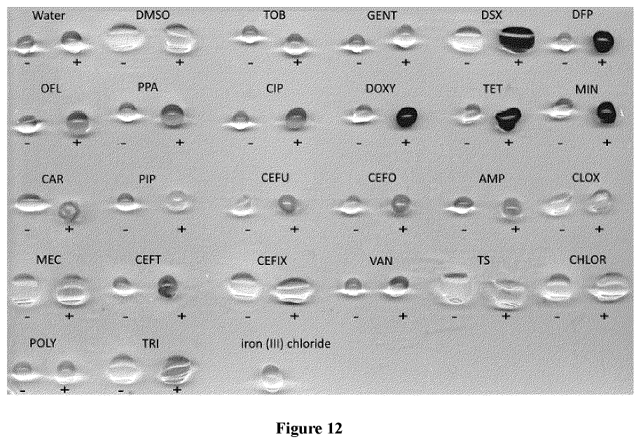

A panel of common antibiotics for potential iron-chelating activity was first using a qualitative assay, monitoring change in colour upon addition of FeCl₃. Binding of transition metals results in formation of coloured complexes that absorb in the visible wavelengths of light, detectable by spectroscopy and by eye. The panel consisted of 22 antibiotics from the aminoglycoside, fluoroquinolone, beta-lactam, and tetracycline classes (FIG. 12). Iron chelators DFP and DSX served as positive controls, turning dark red/violet upon addition of ferric iron at a final concentration of 10 μM. The tetracyclines—doxycycline (DOXY), tetracycline, and minocycline—exhibited similar colour changes. The fluoroquinolones—ciprofloxacin, ofloxacin, and pipemedic acid—formed orange complexes; however, the intensity of the colour change was weaker compared to the tetracyclines, DFP, and DSX. A number of beta-lactams showed colour changes ranging from a brown-orange to red-orange. Ceftriaxone was the only beta-lactam that turned red in the presence of ferric iron. Trimethoprim turned golden-yellow.

### Binding of Ferric Iron Shifts Absorption Spectra

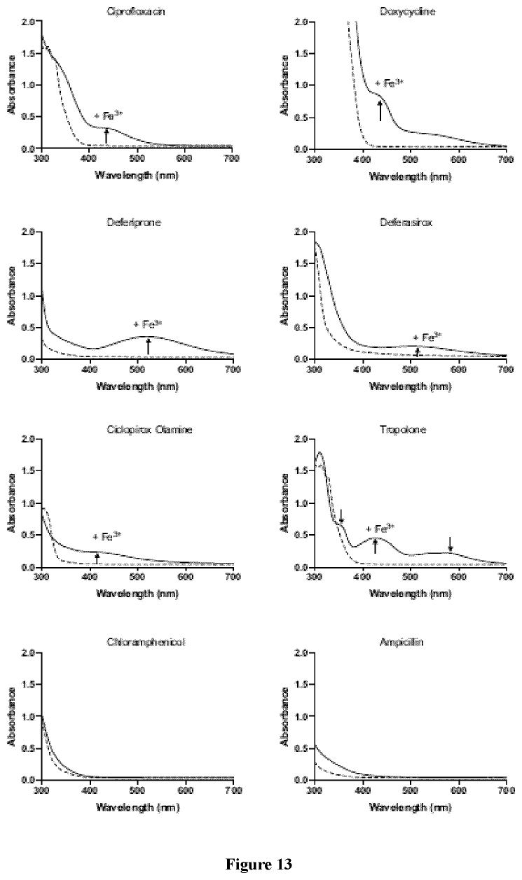

To verify spectral shifts for compounds that changed colour upon addition of ferric iron, a 96-well spectrophotometric assay was performed, with final concentrations of antibiotic and FeCl₃ of 300 μM each. The absorption spectra were scanned from 300-700 nm. The spectra of ciprofloxacin (CIP), pipemedic acid, ofloxacin, tetracycline, minocycline, DOXY, DSX and DFP shifted after the addition of FeCl₃ (FIG. 13), confirming the results of the qualitative assay. Chloramphenicol and ampicillin served as negative controls. The spectrum for ceftriaxone did not change at the concentrations tested, suggesting that the changes in color observed for beta-lactams were likely due to concentration effects.

Identification of Other Compounds that Chelate Iron

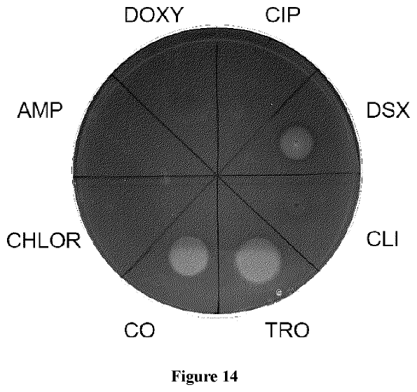

To expand the panel of potential iron inhibitors beyond known antibiotics, we searched the literature for bioactive compounds that were reported to have iron-inhibiting activity. We identified 14 compounds (Table 5) plus gallium nitrate (GN). Gallium is an iron analogue that inhibits siderophore production, iron uptake, and the activity of enzymes that use iron. The spectrophotometric assay was repeated for all compounds listed in Table 5 except for clioquinol (CLI), which was identified in Example 1 as a P. aeruginosa growth inhibitor but precipitated at concentrations above 8 μg/mL. Ciclopirox olamine (CO) and tropolone (TRO) showed shifts in their absorption spectra (FIG. 13). A chrome azurol S (CAS) assay was also used to detect iron binding through de-colourization of the blue agar, indicating removal of Fe³⁺ from the CAS-HDTMA complex (FIG. 14). DSX, TRO, and CO showed the greatest decolourization, and thus the highest relative affinity for iron. Interestingly, DOXY showed a marked colour shift in the presence of Fe³⁺ (FIG. 13) but minimally decolourized CAS agar.

Numerous Iron Inhibitors Synergize with TS

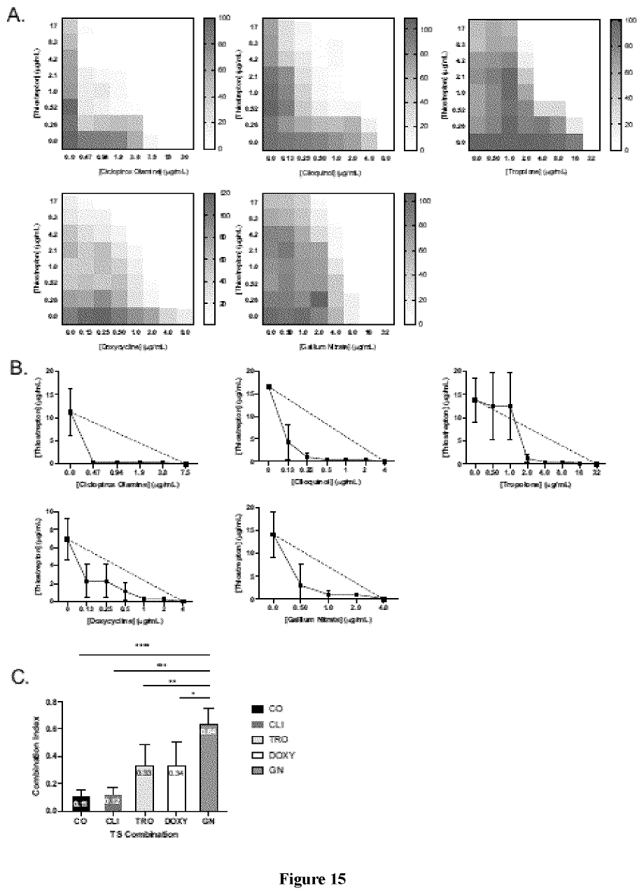

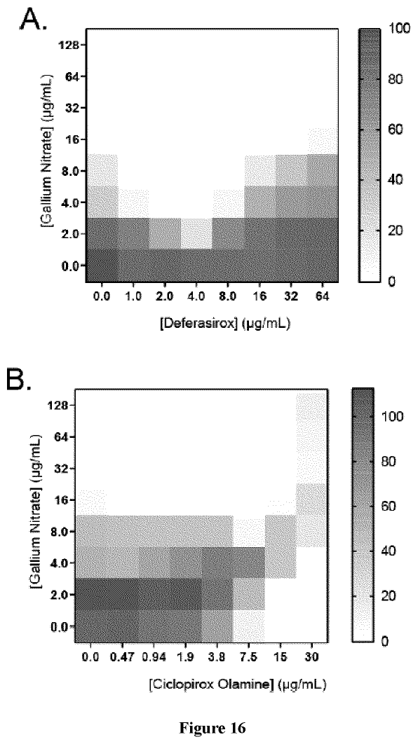

Based on their ability to bind iron, each compound from Table 5, as well as DOXY and CIP, were assessed for synergy with TS using checkerboard assays. DOXY, CO, CLI, TRO, and GN all synergized with TS (FIG. 15), as IC₅₀ isobolograms showed that all combinations were below the line of additivity. Combination indices (CIs) were less than 1 (FIG. 15E). Based on the checkerboards, isobolograms, and CI values, CO and CLI demonstrated the most potent synergy with TS while GN had the weakest. Attempts to combine GN with DSX or CO resulted in antagonism, likely due to the chelators binding Ga³⁺ (FIG. 16).

### Each Compound Potentiates TS Activity

CLI, TRO, DOXY, and CO can inhibit P. aeruginosa growth, suggesting that the innate activity of the compounds could be partly responsible for synergy with TS. Thus, four potential mechanisms of synergy were considered: 1) TS potentiates the activity of each compound through an unknown mechanism; 2) the compound potentiates TS activity by chelating calcium and magnesium and increasing outer membrane permeability or 3) by chelating iron and increasing TS uptake. In all these cases, the synergy is unidirectional. 4) TS and the compound potentiate one another through an unknown mechanism.

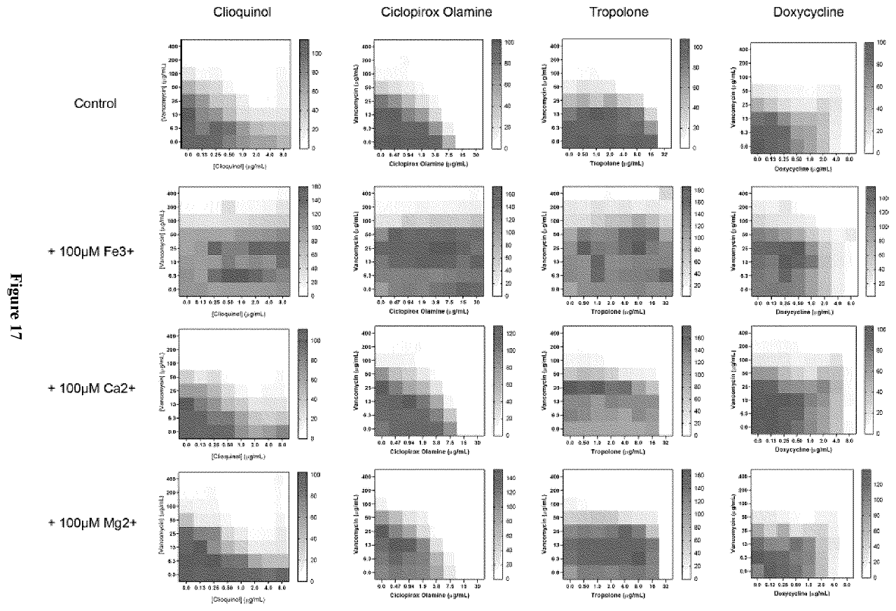

These data suggest that the synergy between TS and each compound is due to their iron chelation capacity rather than membrane permeabilization. First, to determine if DOXY could increase outer membrane permeability, vancomycin (VAN) and DOXY combinations were tested against PA14 alone or in the presence of Ca²⁺, Mg²⁺ or Fe³⁺ (FIG. 17). VAN was selected because it is similar to TS in size but unlike TS, its activity is unrelated to iron availability. VAN has a high minimal inhibitory concentration (MIC) against P. aeruginosa due to limited uptake across the outer membrane. If a compound increases membrane permeability, synergy with VAN is expected. In the checkerboard assays, no synergy was identified for VAN+DOXY, VAN+CLI, VAN+CO, or VAN+TRO. Further, addition of 100 μM Mg²⁺ or Ca²⁺ had no effect on the checkerboard profiles compared to control. In contrast, addition of 100 μM Fe³⁺ abrogated the inhibitory activity of CLI, TRO, and CO, confirming that iron chelation is a critical part of the mechanism by which those compounds impede growth. Lack of synergy between VAN+DOXY also suggested lack of membrane permeabilization. The addition of 100 μM Mg²⁺ had no effect on the checkerboard whereas the addition of Fe³⁺ and Ca²⁺ had a negligible effect. This is reflective of the relatively weak ability of DOXY to compete for iron in the CAS assay (FIG. 14) and of its weak synergy with TS compared to other compounds.

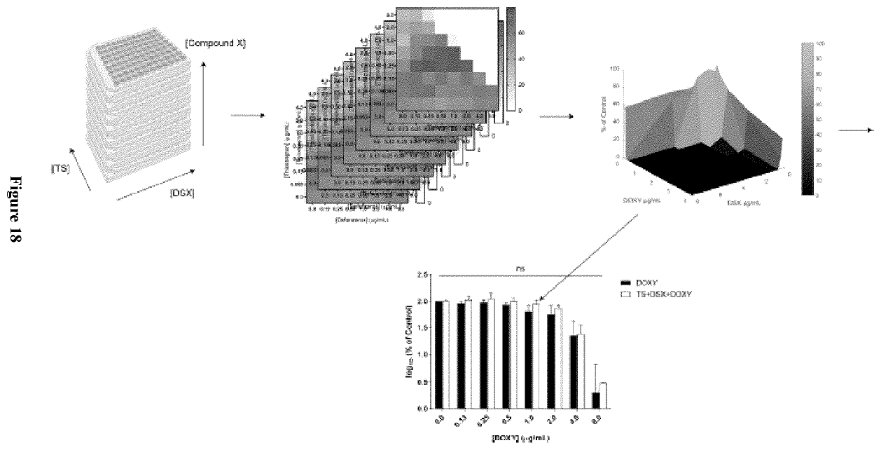

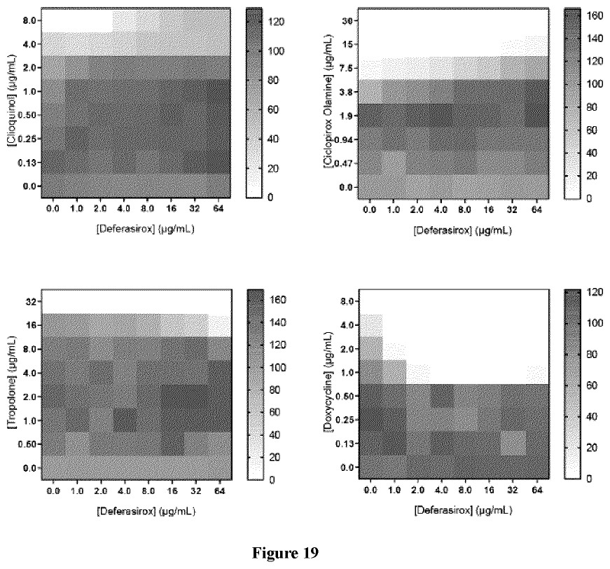

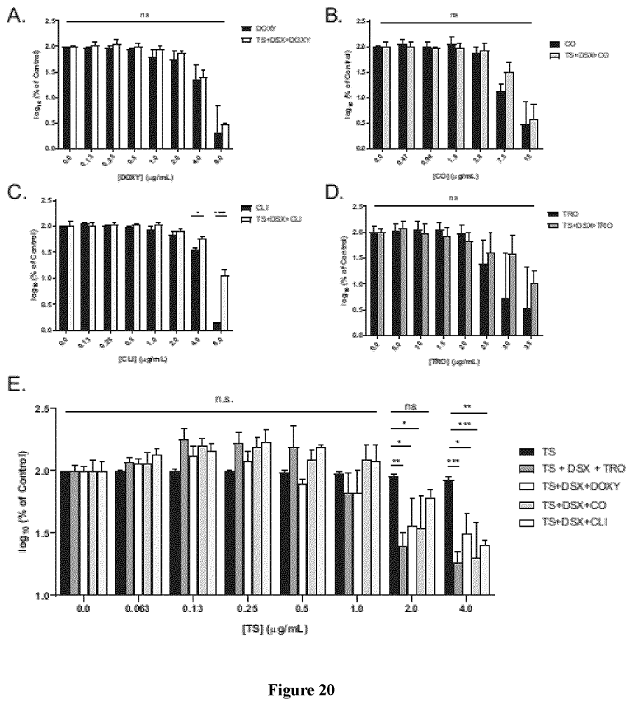

To test the hypothesis that the compounds potentiate TS activity, rather than the other way around, 3D checkerboard assays were performed using PA14. The surface area of each checkerboard was expressed as % of control and graphed against the concentration of the third compound (FIG. 18). Individual MIC assays for each compound were performed and the results graphed as % of control on the same y-axis on a log₁₀ scale. Significant differences between the two datasets would indicate that the TS+DSX combination potentiates the activity of the test compound. To account for potential antagonism between test compounds and DSX, 2D checkerboard assays were conducted (FIG. 19). DSX+TRO and DSX+CO were indifferent. CLI antagonized with DSX at the MIC; however, the concentrations of CLI could not be tested greater than 8 μg/mL due to its poor solubility. DSX was additive with DOXY. No significant differences were observed between the activity of the chelators alone or in combination with TS and DSX, except for with CLI (FIG. 20A to 20D). CLI antagonized DSX at the highest concentration; however, growth was still below 20% of control (FIG. 20C), equivalent to the MIC in the growth medium. When the data were plotted against TS concentration (FIG. 20E), significant differences for the combinations were apparent at 2 and 4 μg/mL TS compared to TS alone, indicating that the compounds and DSX potentiate TS activity. These data suggest that the synergy between the chelators and TS is unidirectional.

### TS Combinations are Bactericidal and Effective Against Clinical Isolates

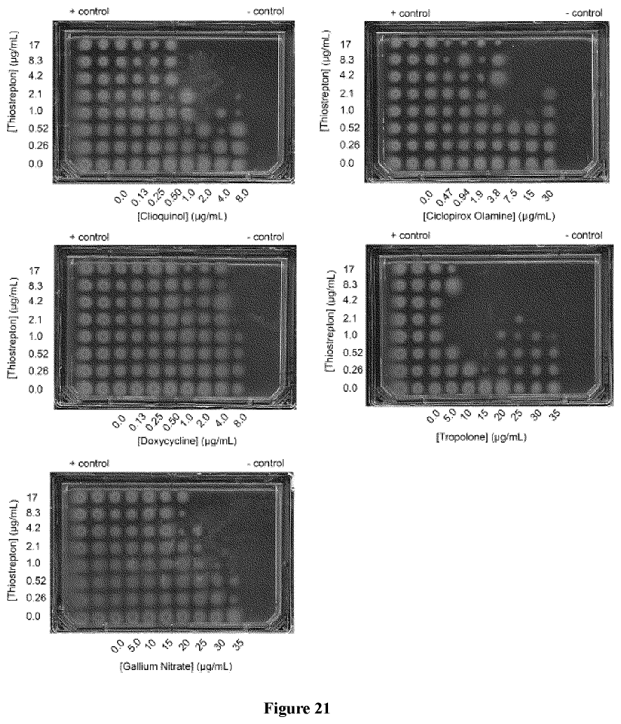

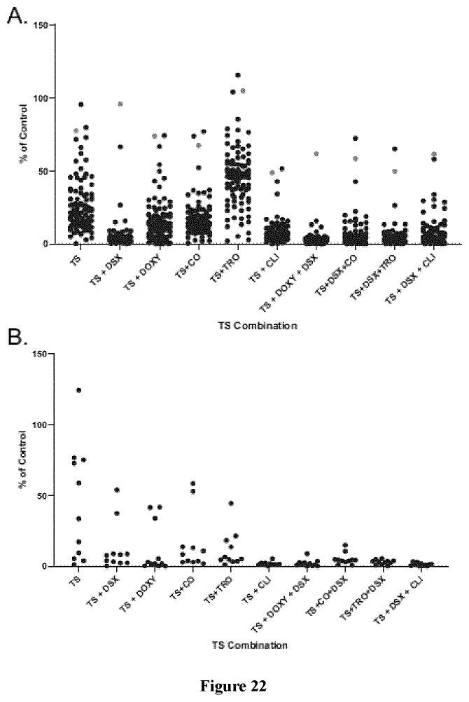

TS, CO, CLI, DOXY, and TRO alone were bacteriostatic; however, when combined with TS, the combinations were bactericidal (FIG. 21). This improved activity prompted the testing of double (TS+compound) and triple combinations (TS+DSX+compound) against same panels of P. aeruginosa and A. baumannii clinical isolates assayed for susceptibility to TS+DSX (FIG. 22). GN was omitted due to its weak synergy with TS against PA14 and antagonism with iron chelators (FIG. 16). TS and DSX were used at 8.3 μg/mL (5 μM) and 32 μg/mL as before, while the other compounds were added at ⅛^{th} the MIC of PA14, corresponding to DOXY, CO, TRO, and CLI concentrations of 1 μg/mL, 2 μg/mL, 4 μg/mL, and 1 μg/mL, respectively.

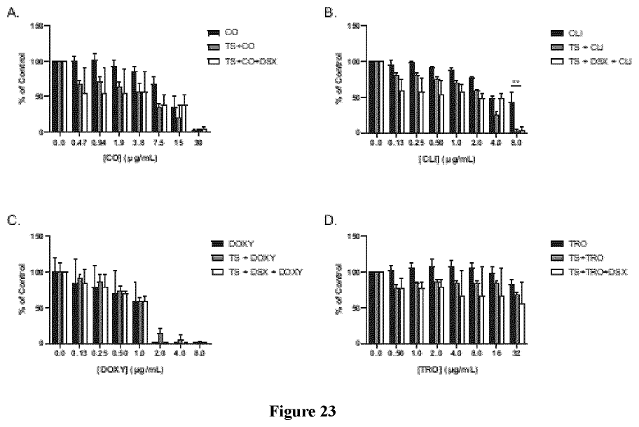

Of the double combinations, TS+DSX was the most potent against P. aeruginosa (FIG. 22A), consistent with our checkerboard assays. Interestingly, TS+DOXY and TS+CO had similar potency despite differences in their CI values (FIG. 15). TS+TRO was the least potent of the double combinations. TS+CLI potency was similar to TS+DSX, and this combination reduced growth of our most resistant clinical isolate, C0379, while TS+DSX did not. TS synergized with CLI to inhibit C0379 although a higher concentration (8 μg/mL) of CLI was required (FIG. 23). Of the triple combinations, TS+DSX+DOXY was the most potent, with only C0379 showing resistance. We previously reported that C0379 has a partial deletion of fpvB, encoding a pyoverdine receptor (22). However, triple combinations with higher concentrations of DOXY and CO could inhibit its growth (FIGS. 23A and 23C). C0379 growth was also inhibited by TS+CLI or TS+DSX+CLI, if CLI was used at 8 μg/mL. CLI alone did not reduce growth below MIC and there was no antagonism between DSX and CLI with C0379 compared to PA14 (FIG. 23B). C0379 was also less susceptible to TRO compared to PA14 (FIG. 23D).

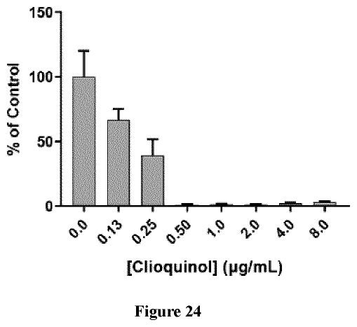

For A. baumannii isolates, all double combinations were equally effective. TS+CLI was highly potent against A. baumannii compared to P. aeruginosa when CLI was used at 1 μg/mL (FIG. 22B and FIG. 24). Strain C0286 was resistant to TS but susceptible to TS+CLI, suggesting inhibition was due to CLI. Conversely, TS+TRO had little activity against P. aeruginosa clinical isolates but was effective against A. baumannii. The triple combinations inhibited the growth of both species.

### Discussion

Herein the inventors identified multiple compounds that synergize with TS against P. aeruginosa and A. baumannii clinical isolates, due to their ability to chelate iron. Iron-binding capacity was demonstrated by monitoring visual color changes when complexed with Fe³⁺, CAS agar decolorization, and via spectrophotometric assays. The CAS assay, which is used to detect siderophore production, not only indicates whether a compound can bind iron, but also if it has a stronger affinity for the metal than the CAS-HDTMA complex. This allowed us to compare the relative binding affinities of various compounds based on the extent of decolourization. This method is limited by compound solubility, as seen with CLI (FIG. 14).

None of the natural phytochelators from plants that were tested—including baicalin, ferulic acid, sodium phytate, 2,3,5,6-tetrametylpyrazine, curcumin, epigallocatechin gallate, and phloretin (Table 5)—synergized with TS. P. aeruginosa can act as a plant pathogen and may have evolved to outcompete or even take up phytochelators. The compounds that synergized with TS are all synthetic and the extent of synergy correlated with their ability to strip iron from CAS-Fe³⁺-HDTMA complexes (FIG. 15 and FIG. 14). Iron chelators compete with siderophores and reduce iron availability, resulting in increased pyoverdine receptor expression and susceptibility to TS. Weaker chelators such as DOXY and CIP showed little or no synergy with TS whereas strong chelators like CO and TRO exhibited greater synergy.

The GN data demonstrate that synergy with TS can occur via routes other than iron chelation. Ga³⁺ represses pyoverdine production and forms complexes with pyoverdine that prevents iron binding. TS activity could be weakly potentiated because of reduced competition for pyoverdine receptors if siderophore production decreases upon GN treatment. These data show that disrupting iron acquisition may be another avenue for novel TS combinations. GN in triple combinations with TS+chelator has limited utility because iron chelators bind Ga³⁺ (FIG. 16).

In summary, TS synergizes with iron-chelating compounds of diverse structure that were not primarily intended as antibacterial compounds. For example, CLI has antifungal and antiprotozoal properties and was investigated as a potential treatment for Alzheimer's Disease; however, the compound was shown to be neurotoxic at higher concentrations. CO is also used as a topical antifungal agent. Given that P. aeruginosa is a burn wound pathogen, and the precedence of these chelators as topical agents, the combinations shown herein may be useful in treating superficial infections caused by this pathogen. Although the mechanisms of action for some of these molecules are not fully understood, they also reveal new targets for antibiotic therapy. In addition, TS combinations demonstrated bactericidal activity while chelator compounds alone were bacteriostatic. The new combinations were effective against clinical isolates resistant to TS+DSX. The combinations were more potent against A. baumannii isolates than those of P. aeruginosa (FIG. 22). It is possible that there are differences in pyoverdine receptor density or affinity for TS between the two species that lead to increased uptake by A. baumannii. To keep the concentrations of the chelators in the combinations consistent, ⅛^{th} the MIC against P. aeruginosa PA14 was used. It is possible that the MICs of the A. baumannii clinical isolates were intrinsically lower. Our data suggests that these compounds have dual roles—as antibacterial agents and TS adjuvants. Iron restriction mimics many in vivo conditions, as host proteins sequester free iron in an attempt to starve bacteria and exert antibacterial activity. Screening for antibiotic activity under similar conditions is an important strategy for development of new treatments for the most dangerous pathogens.

### Example 3. A Combination of Thiopeptides and Iron Chelators are Effective in Inhibiting P. aeruginosa and Acinetobacter baumanii

Further prompted by the ability for thiostrepton (TS), a gram-positive thiopeptide antibiotic, to synergize with various iron chelators to inhibit the growth of gram-negative bacterial isolates, other thiopeptides were tested for synergy in combination with DSX.

### Methods

### Dose Response and Checkerboard Assays

Culture conditions, checkerboard assays using P. aeruginosa PA14 and dose-response assays were performed as described in Example 1. In addition to TS, other thiopeptides tested were siomycin A (SM), thiocillin I (TC), micrococcin P1 (MC), nosiheptide (NH), berninamycin A (BER), and geninthiocin A (GEN). For dose response assays of the combinations conducted in 10:90, DSX concentration was constant at 64 μg/mL.

### Results

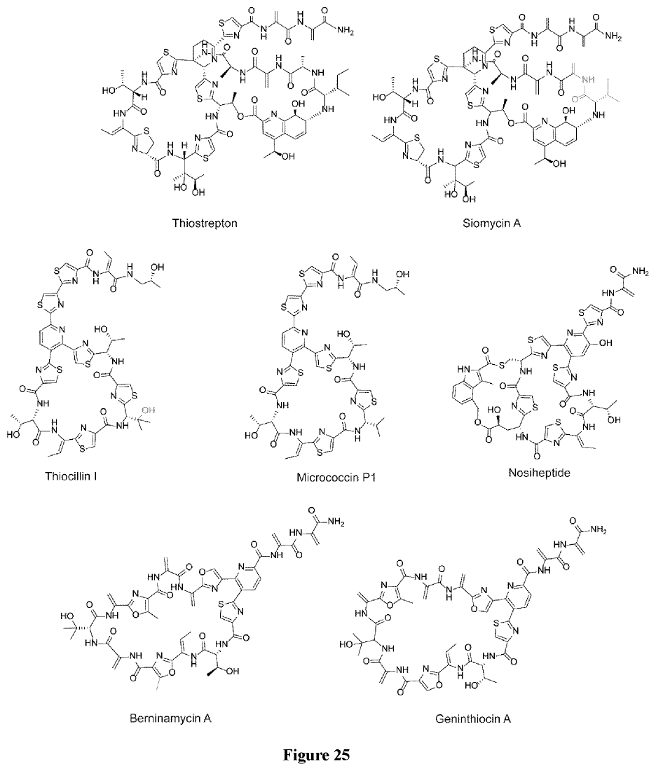

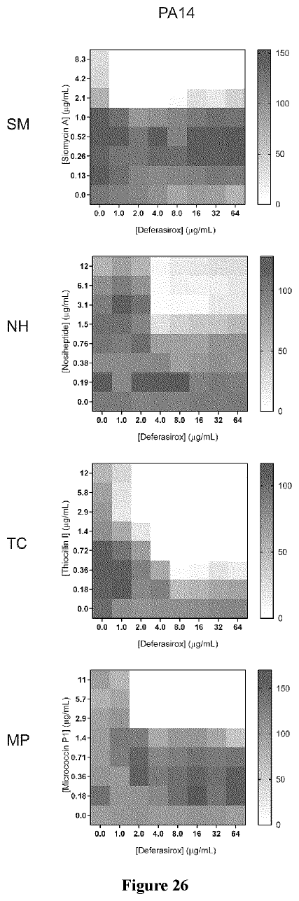

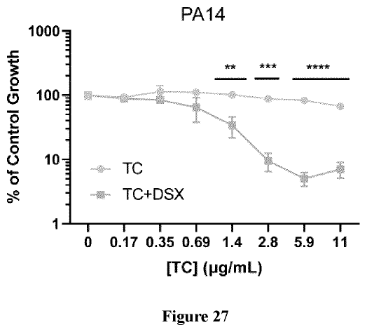

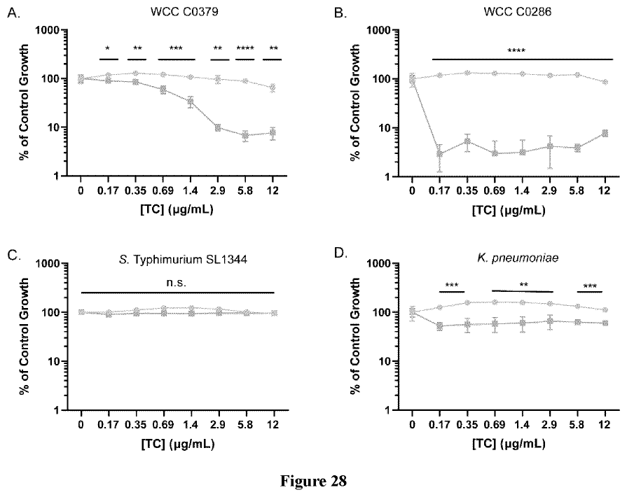

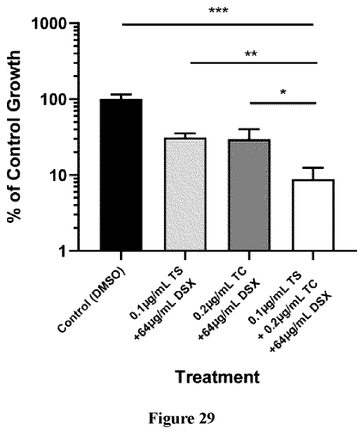

The structures of thiopeptides tested for synergy with DSX, including TS, are shown in FIG. 25. The following are analogue pairs: TS & SM, TC & MC, and BER & GEN. No structural analogues of NH were tested. Checkerboard assays show the thiopeptides, SM, NH, TC and MP synergize with DSX with varying activity against P. aeruginosa (FIG. 26). Thiopeptides BER and GEN are not shown as they did not synergize with DSX. Dose-response assays of TC versus TC+DSX against PA14 was assessed, demonstrating increased inhibition of the combination (FIG. 27). Following this, TC and TC+DSX dose-response assays were then compared against various bacteria (FIG. 28). These data show that gram-negative P. aeruginosa WCC C0379 (TS-resistant) and A. baumannii C0286 are susceptible to the dual combination. The triple combination of TS+TC+DSX at sub-MIC reduces PA14 growth below the MIC (FIG. 29). These data reveal the ability to tailor combinations of thiopeptides and iron chelators for different clinical isolates and resistant strains.

While the present application has been described with reference to examples, it is to be understood that the scope of the claims should not be limited by the embodiments set forth in the examples, but should be given the broadest interpretation consistent with the description as a whole.

### REFERENCES

1. Zabawa T P, Pucci M J, Parr T R, Jr., Lister T. 2016. Treatment of Gram-negative bacterial infections by potentiation of antibiotics. Curr Opin Microbiol 33:7-12.
2. Blair J M, Webber M A, Baylay A J, Ogbolu D O, Piddock U. 2015. Molecular mechanisms of antibiotic resistance. Nat Rev Microbiol 13:42-51.
3. Hall C W, Mah T F. 2017. Molecular mechanisms of biofilm-based antibiotic resistance and tolerance in pathogenic bacteria. FEMS Microbiol Rev 41:276-301.
4. Kalan L, Wright G D. 2011. Antibiotic adjuvants: multicomponent anti-infective strategies. Expert Rev Mol Med 13:e5.
5. Tacconelli E, Carrara E, Savoldi A, Harbarth S, Mendelson M, Monnet D L, Pulcini C, Kahlmeter G, Kluytmans J, Carmeli Y, Ouellette M, Outterson K, Patel J, Cavaleri M, Cox E M, Houchens C R, Grayson M L, Hansen P, Singh N, Theuretzbacher U, Magrini N, Group WHOPPLW. 2018. Discovery, research, and development of new antibiotics: the WHO priority list of antibiotic-resistant bacteria and tuberculosis. Lancet Infect Dis 18:318-327.
6. Fothergill J L, Winstanley C, James C E. 2012. Novel therapeutic strategies to counter Pseudomonas aeruginosa infections. Expert Rev Anti Infect Ther 10:219-35.
7. Rybtke M, Hultqvist L D, Givskov M, Tolker-Nielsen T. 2015. Pseudomonas aeruginosa Biofilm Infections: Community Structure, Antimicrobial Tolerance and Immune Response. J Mol Biol 427:3628-45.
8. Burrows L L. 2018. The Therapeutic Pipeline for Pseudomonas aeruginosa Infections. ACS Infect Dis 4:1041-1047.
9. Nguyen L, Garcia J, Gruenberg K, MacDougall C. 2018. Multidrug-Resistant Pseudomonas Infections: Hard to Treat, But Hope on the Horizon? Curr Infect Dis Rep 20:23.
10. Page M G, Heim J. 2009. Prospects for the next anti-Pseudomonas drug. Curr Opin Pharmacol 9:558-65.
11. Wenderska I B, Chong M, McNulty J, Wright G D, Burrows L L. 2011. Palmitoyl-D L-carnitine is a multitarget inhibitor of Pseudomonas aeruginosa biofilm development. Chembiochem 12:2759-66.
12. Ejim L, Farha M A, Falconer S B, Wildenhain J, Coombes B K, Tyers M, Brown E D, Wright G D. 2011. Combinations of antibiotics and nonantibiotic drugs enhance antimicrobial efficacy. Nat Chem Biol 7:348-50.
13. Delcour A H. 2009. Outer membrane permeability and antibiotic resistance. Biochim Biophys Acta 1794:808-16.
14. Cox G, Wright G D. 2013. Intrinsic antibiotic resistance: mechanisms, origins, challenges and solutions. Int J Med Microbiol 303:287-92.
15. Liberati N T, Urbach J M, Miyata S, Lee D G, Drenkard E, Wu G, Villanueva J, Wei T, Ausubel F M. 2006. An ordered, nonredundant library of Pseudomonas aeruginosa strain PA14 transposon insertion mutants. Proc Natl Acad Sci USA 103:2833-8.
16. Maier L, Pruteanu M, Kuhn M, Zeller G, Telzerow A, Anderson E E, Brochado A R, Fernandez K C, Dose H, Mori H, Patil K R, Bork P, Typas A. 2018. Extensive impact of non-antibiotic drugs on human gut bacteria. Nature 555:623-628.
17. Anderson B, Hodgkin D C, Viswamitra M A. 1970. The structure of thiostrepton. Nature 225:233-5.
18. Jonker H R, Baumann S, Wolf A, Schoof S, Hiller F, Schulte K W, Kirschner K N, Schwalbe H, Arndt H D. 2011. NMR structures of thiostrepton derivatives for characterization of the ribosomal binding site. Angew Chem Int Ed Engl 50:3308-12.
19. Bleich R, Watrous J D, Dorrestein P C, Bowers A A, Shank E A. 2015. Thiopeptide antibiotics stimulate biofilm formation in Bacillus subtilis. Proc Natl Acad Sci USA 112:3086-91.

1-50. (canceled)

51. A combination comprising a thiopeptide antibiotic and an iron inhibitor.

52. The combination of claim 51, wherein the thiopeptide antibiotic is thiostrepton, siomycin A, thiocillin I, micrococcin P1, nosiheptide, berninamycin A, geninthiocin A, a derivative thereof, a prodrug thereof, a salt thereof, or a combination thereof.

53. The combination of claim 51, wherein the iron inhibitor is an iron chelator or an iron analogue.

54. The combination of claim 51, wherein the iron inhibitor comprises deferiprone, deferasirox, deferoxamine, transferrin, hemoglobin, lactoferrin, doxycycline, ciclopirox olamine, tropolone, clioquinol, gallium nitrate, or a combination thereof.

55. The combination of claim 51, wherein the thiopeptide antibiotic and the iron inhibitor are in synergistic amounts for treating and/or preventing a bacterial infection in a subject.

56. The combination of claim 51, wherein the bacterial infection is a gram-negative bacterial infection.

57. The combination of claim 56, wherein the bacterial infection is a Pseudomonas aeruginosa infection, an Acinetobacter baumannii infection, or a combination thereof.

58. The combination of claim 51, wherein the bacterial infection is a multi-drug resistant bacterial infection.

59. The combination of claim 58, wherein the bacterial infection is caused by a bacteria that expresses a siderophore receptor.

60. The combination of claim 51, wherein the bacterial infection is caused by a bacteria that expresses a type I pyoverdine receptor, wherein the type I pyoverdine receptor is FpvA, FpvB, a homolog thereof, or a combination thereof.

61. A composition or kit comprising the combination of claim 51.

62. A method of treating and/or preventing a gram-negative bacterial infection in a subject, the method comprising administering a thiopeptide antibiotic to the subject.

63. The method of claim 62, wherein the thiopeptide antibiotic is thiostrepton, siomycin A, thiocillin I, micrococcin P1, nosiheptide, berninamycin A, geninthiocin A, a derivative thereof, a prodrug thereof, a salt thereof, or a combination thereof.

64. The method of claim 62, wherein the method further comprises administering an iron inhibitor to the subject.

65. The method of claim 62, wherein the iron inhibitor comprises deferiprone, deferasirox, deferoxamine, transferrin, hemoglobin, lactoferrin, doxycycline, ciclopirox olamine, tropolone, clioquinol, gallium nitrate, or a combination thereof.

66. The method of claim 62, wherein the thiopeptide antibiotic and the iron inhibitor synergistically treat and/or prevent the bacterial infection.

67. The method of claim 62, wherein the bacterial infection is a Pseudomonas aeruginosa infection, an Acinetobacter baumannii infection, or a combination thereof.

68. The method of claim 62, wherein the bacterial infection is a multi-drug resistant bacterial infection.

69. The method of claim 62, wherein the bacterial infection is caused by a bacteria that expresses a siderophore receptor.

70. The method of claim 62, wherein the bacterial infection is caused by a bacteria that expresses a type I pyoverdine receptor, wherein the type I pyoverdine receptor is FpvA, FpvB, a homolog thereof, or a combination thereof.


SEQUENCE LISTING (14 sequences)

SEQ ID NO: 1 - DNA, 28 bp, Artificial Sequence
  Primer
gaatcccgggcggtaggacgaccatgac

SEQ ID NO: 2 - DNA, 28 bp, Artificial Sequence
  Primer
cttcaagcttttatcggttggccgcgag

SEQ ID NO: 3 - DNA, 30 bp, Artificial Sequence
  Primer
cgttggaattcctggaccggcctgtcctac

SEQ ID NO: 4 - DNA, 30 bp, Artificial Sequence
  Primer
ggccggagctccgccgcgccggtgttctgc

SEQ ID NO: 5 - DNA, 30 bp, Artificial Sequence
  Primer
cgctcgagctcgttcaccgcgcagcagcaa

SEQ ID NO: 6 - DNA, 30 bp, Artificial Sequence
  Primer
cggccaagcttaatcggccgccggaagtcg

SEQ ID NO: 7 - DNA, 30 bp, Artificial Sequence
  Primer
gccggaattcggctacgagtggaccggcct

SEQ ID NO: 8 - DNA, 30 bp, Artificial Sequence
  Primer
ctccgagctcgtcggccaccggcgcggcgg

SEQ ID NO: 9 - DNA, 30 bp, Artificial Sequence
  Primer
cgatgagctccgcagcagcttcctcaacga

SEQ ID NO: 10 - DNA, 29 bp, Artificial Sequence
  Primer
tctcgaagcttcagcgccaaccccgtcgt

SEQ ID NO: 11 - DNA, 29 bp, Artificial Sequence
  Primer
gactggaattccgactcgcggcaatacac

SEQ ID NO: 12 - DNA, 29 bp, Artificial Sequence
  Primer
cgctggagctcggggccgacgctgcaggc

SEQ ID NO: 13 - DNA, 30 bp, Artificial Sequence
  Primer
accagagctccgcgaggaactggcgcaggc

SEQ ID NO: 14 - DNA, 29 bp, Artificial Sequence
  Primer
cctgaagcttgctgcccggatgccggccg